SEQUENCE LISTING (42 sequences)

SEQ ID NO: 1 - DNA, 21 bp, Artificial
  DNA primer
tgctgtctttgctgtcctgat

SEQ ID NO: 2 - DNA, 28 bp, Artificial
  DNA primer
atatgcggccgcgggggggggggggggg

SEQ ID NO: 3 - DNA, 21 bp, Artificial
  DNA primer
ttgaccaggcatcccagggtc

SEQ ID NO: 4 - DNA, 21 bp, Artificial
  DNA primer
caagaagcacacgactgaggc

SEQ ID NO: 5 - DNA, 30 bp, Artificial
  DNA primer
ggtgtgcattccatggactggacctggagg

SEQ ID NO: 6 - DNA, 366 bp, Artificial
  2G11 VH nucleotide sequence
gaggtgaagctggtggagtcaggacctagcctcgtgaaaccttctcagactctgtccctcacctgttctgtcacgggcgactccatcaccagtggttattggaactggatccggaaattcccagggaaaaaacttgaatacatggggttcataagctacagtggtgacacttattacaatccatctctcaaaagtcgaatctccatcactcgagacacatccaagaaccagtaccacctgcagttgaattctgtgacttctgaggacacagcaacatattactgtgcaagaaggaactacgttagtaactacggggggtggtacttcggtgtctggggcgcagggaccacggtcaccgtctcctca

SEQ ID NO: 7 - protein, 122 aa, Artificial
  2G11 VH amino acid sequence
EVKLVESGPSLVKPSQTLSLTCSVTGDSITSGYWNWIRKFPGKKLEYMGFISYSGDTYYNPSLKSRISITRDTSKNQYHLQLNSVTSEDTATYYCARRNYVSNYGGWYFGVWGAGTTVTVSS

SEQ ID NO: 8 - DNA, 321 bp, Artificial
  2G11 VL nucleotide sequence
aacattgtaatgacccaatctcccaaatccatgtccatgtcagtaggagagagagtcaccctgagctgcaaggccagtgagaatgtggttacttatgtatcctggtatcaacagaaaccagatcagtctcctaaactgctgatatacggggcatccaaccggtacactggggtccctgatcgcttcacaggcagtggatctgcaacagatttcactcttatcatcagcagtgttcaggctgaagaccttgcagattatcactgtggacagagttacacctatcctcccacgttcggtgctgggaccaagctggagctgaaa

SEQ ID NO: 9 - protein, 107 aa, Artificial
  2G11 VL amino acid sequence
NIVMTQSPKSMSMSVGERVTLSCKASENVVTYVSWYQQKPDQSPKLLIYGASNRYTGVPDRFTGSGSATDFTLIISSVQAEDLADYHCGQSYTYPPTFGAGTKLELK

SEQ ID NO: 10 - DNA, 732 bp, Artificial
  2G11 ScFv nucleotide sequence
aacattgtaatgacccaatctcccaaatccatgtccatgtcagtaggagagagagtcaccctgagctgcaaggccagtgagaatgtggttacttatgtatcctggtatcaacagaaaccagatcagtctcctaaactgctgatatacggggcatccaaccggtacactggggtccctgatcgcttcacaggcagtggatctgcaacagatttcactcttatcatcagcagtgttcaggctgaagaccttgcagattatcactgtggacagagttacacctatcctcccacgttcggtgctgggaccaagctggagctgaaaggtggaggcggatctggcggaggtggctctggcggtggcggatcggaggtgaagctggtggagtcaggacctagcctcgtgaaaccttctcagactctgtccctcacctgttctgtcacgggcgactccatcaccagtggttattggaactggatccggaaattcccagggaaaaaacttgaatacatggggttcataagctacagtggtgacacttattacaatccatctctcaaaagtcgaatctccatcactcgagacacatccaagaaccagtaccacctgcagttgaattctgtgacttctgaggacacagcaacatattactgtgcaagaaggaactacgttagtaactacggggggtggtacttcggtgtctggggcgcagggaccacggtcaccgtctcctca

SEQ ID NO: 11 - protein, 244 aa, Artificial
  2G11 ScFv coding amino acid sequence followed by linker and then 2G11 VH amino acid sequence
NIVMTQSPKSMSMSVGERVTLSCKASENVVTYVSWYQQKPDQSPKLLIYGASNRYTGVPDRFTGSGSATDFTLIISSVQAEDLADYHCGQSYTYPPTFGAGTKLELKGGGGSGGGGSGGGGSEVKLVESGPSLVKPSQTLSLTCSVTGDSITSGYWNWIRKFPGKKLEYMGFISYSGDTYYNPSLKSRISITRDTSKNQYHLQLNSVTSEDTATYYCARRNYVSNYGGWYFGVWGAGTTVTVSS

SEQ ID NO: 12 - protein, 447 aa, Artificial
  Hinge h1+hinge h4, Gly-Ser linker and Gly-Leu linker iand hCH3 IgG3 domains
GAGCTCAAAACCCCACTTGGTGACACAACTCACACAGAGCCCAAATCTTGTGACACACCTCCCCCGTGCCCAAGGTGCCCAGGCGGTGGAAGCAGCGGAGGTGGAAGTGGAGGACAGCCCCGAGAACCACAGGTGTACACCCTGCCCCCATCCCGGGAGGAGATGACCAAGAACCAGGTCAGCCTGACCTGCCTGGTCAAAGGCTTCTACCCCAGCGACATCGCCGTGGAGTGGGAGAGCAGCGGGCAGCCGGAGAACAACTACAACACCACGCCTCCCATGCTGGACTCCGACGGCTCCTTCTTCCTCTACAGCAAGCTCACCGTGGACAAGAGCAGGTGGCAGCAGGGGAACATCTTCTCATGCTCCGTGATGCATGAGGCTCTGCACAACCGCTTCACGCAGAAGAGCCTCTCCCTGTCTCCGGGTAAAGGCCTCGGTGGCCTG

SEQ ID NO: 13 - protein, 149 aa, Artificial
  Hinge h1, Hinge h4, linker and hCH3 IgG3 domains
ELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGL

SEQ ID NO: 14 - protein, 12 aa, Artificial
  Hinge regions (IgG3 upper hinge)
ELKTPLGDTTHT

SEQ ID NO: 15 - protein, 15 aa, Artificial
  Hinge region (IgG3, lower hinge, 15 amino acids)
EPKSCDTPPPCPRCP

SEQ ID NO: 16 - protein, 10 aa, Artificial
  Gly-Ser Linker
GGGSSGGGSG

SEQ ID NO: 17 - protein, 107 aa, Artificial
  hCH3 IgG3
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 5 aa, Artificial
  Linker
GLGGL

SEQ ID NO: 19 - protein, 15 aa, Artificial
  Linker
GGGGSGGGGSGGGGS

SEQ ID NO: 20 - protein, 12 aa, Artificial
  Upper hinge hIgG3 (h1)
ELKTPLGDTTHT

SEQ ID NO: 21 - protein, 15 aa, Artificial
  Lower hinge hIgG3 (h4)
EPKSCDTPPPCPRCP

SEQ ID NO: 22 - protein, 107 aa, Artificial; identical to SEQ ID NO: 17
  CH3 hIgG3

SEQ ID NO: 23 - protein, 350 aa, Artificial
  glycoprotein D from Herpes Simplex virus 2 with an optional His-tag
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLITGHHHHHH

SEQ ID NO: 24 - protein, 743 aa, Artificial
  Amino acid sequence of suitable chicken vaccibody construct Targeting unit anti-chicken MHC II scFv, dimerization unit human shortened hinge (h1+h4), antigenic unit gD from herpes simplex virus 2 + an optional 6xHis-tag
NIVMTQSPKSMSMSVGERVTLSCKASENVVTYVSWYQQKPDQSPKLLIYGASNRYTGVPDRFTGSGSATDFTLIISSVQAEDLADYHCGQSYTYPPTFGAGTKLELKGGGGSGGGGSGGGGSEVKLVESGPSLVKPSQTLSLTCSVTGDSITSGYWNWIRKFPGKKLEYMGFISYSGDTYYNPSLKSRISITRDTSKNQYHLQLNSVTSEDTATYYCARRNYVSNYGGWYFGVWGAGTTVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLITGHHHHHH

SEQ ID NO: 25 - protein, 94 aa, Artificial
  Chicken IgY CH2
HPSSCTPSQSESVELLCLVTGFSPASAEVEWLVDGVGGLLVASQSPAVRSGSTYSLSSRVNVSGTDWREGKSYSCRVRHPATNTVVEDHVKGCP

SEQ ID NO: 26 - protein, 118 aa, Artificial
  Chicken IgY CH4
GPTTPPLIYPFAPHPEELSLSRVTLSCLVRGFRPRDIEIRWLRDHRAVPATEFVTTAVLPEERTANGAGGDGDTFFVYSKMSVETAKWNGGTVFACMAVHEALPMRFSQRTLQKQAGK

SEQ ID NO: 27 - protein, 10 aa, Artificial
  Upper CH2 sequence IgY
EWLVDGVGGL

SEQ ID NO: 28 - protein, 26 aa, Artificial
  Lower CH2 sequence IgY
EGKSYSCRVRHPATNTVVEDHVKGCP

SEQ ID NO: 29 - protein, 33 aa, Artificial
  Extended lower CH2 sequence IgY
VSGTDWREGKSYSCRVRHPATNTVVEDHVKGCP

SEQ ID NO: 30 - protein, 527 aa, Artificial
  Shortened HA5 with polybasic deletion from influenza A/VietNam 1203/04 H5N1
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSL

SEQ ID NO: 31 - protein, 940 aa, Artificial
  Amino acid sequence of suitable chicken vaccibody construct Targeting unit anti-chicken MHC II, dimerization unit shortened CH2 (lower + upper CH2) + entire CH4, antigenic unit H5 from influenza
NIVMTQSPKSMSMSVGERVTLSCKASENVVTYVSWYQQKPDQSPKLLIYGASNRYTGVPDRFTGSGSATDFTLIISSVQAEDLADYHCGQSYTYPPTFGAGTKLELKGGGGSGGGGSGGGGSEVKLVESGPSLVKPSQTLSLTCSVTGDSITSGYWNWIRKFPGKKLEYMGFISYSGDTYYNPSLKSRISITRDTSKNQYHLQLNSVTSEDTATYYCARRNYVSNYGGWYFGVWGAGTTVTVSSEWLVDGVGGLEGKSYSCRVRHPATNTVVEDHVKGCPGGGSSGGGSGGPTTPPLIYPFAPHPEELSLSRVTLSCLVRGFRPRDIEIRWLRDHRAVPATEFVTTAVLPEERTANGAGGDGDTFFVYSKMSVETAKWNGGTVFACMAVHEALPMRFSQRTLQKQAGKGLGGLDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSL

SEQ ID NO: 32 - protein, 937 aa, Artificial
  Variant 2 with an extended lower CH2 sequence
NIVMTQSPKSMSMSVGERVTLSCKASENVVTYVSWYQQKPDQSPKLLIYGASNRYTGVPDRFTGSGSATDFTLIISSVQAEDLADYHCGQSYTYPPTFGAGTKLELKGGGGSGGGGSGGGGSEVKLVESGPSLVKPSQTLSLTCSVTGDSITSGYWNWIRKFPGKKLEYMGFISYSGDTYYNPSLKSRISITRDTSKNQYHLQLNSVTSEDTATYYCARRNYVSNYGGWYFGVWGAGTTVTVSSVSGTDWREGKSYSCRVRHPATNTVVEDHVKGCPGGGSSGGGSGGPTTPPLIYPFAPHPEELSLSRVTLSCLVRGFRPRDIEIRWLRDHRAVPATEFVTTAVLPEERTANGAGGDGDTFFVYSKMSVETAKWNGGTVFACMAVHEALPMRFSQRTLQKQAGKGLGGLDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSL

SEQ ID NO: 33 - protein, 3039 aa, Artificial
  Infectious bursal disease virus viral protein 2, viral protein 4 and viral protein 3 of segment A (VP2-VP4-VP3)
ATGACAAACCTGCAAGATCAAACCCAACAGATTGTTCCGTTCATACGGAGCCTTCTGATGCCAACAACCGGACCGGCGTCCATTCCGGACGACACCCTAGAGAAGCACACTCTCAGGTCAGAGACCTCGACCTACAATTTGACTGTGGGGGACACAGGGTCAGGGCTAATTGTCTTTTTCCCTGGTTTCCCTGGCTCAATTGTGGGTGCTCACTACACACTGCAGAGCAATGGGAACTACAAGTTCGATCAGATGCTCCTGACTGCCCAGAACCTACCGGCCAGCTACAACTACTGCAGGCTAGTGAGTCGGAGTCTCACAGTGAGGTCAAGCACACTCCCTGGTGGCGTTTATGCACTAAATGGCACCATAAACGCCGTGACCTTCCAAGGAAGCCTGAGTGAACTGACAGATGTTAGCTACAATGGGTTGATGTCTGCAACAGCCAACATCAACGACAAAATCGGGAACGTCCTAGTAGGGGAAGGGGTAACCGTCCTCAGCTTACCCACATCATATGATCTTGGGTATGTGAGACTCGGTGACCCCATTCCCGCTATAGGGCTCGACCCAAAAATGGTAGCAACATGTGACAGCAGTGACAGGCCCAGAGTCTACACCATAACTGCAGCCGATGATTACCAATTCTCATCACAGTACCAGGCAGGTGGGGTAACAATCACACTGTTCTCAGCTAATATCGATGCCATCACAAGCCTCAGCATTGGGGGAGAACTCGTGTTCCAAACAAGCGTCCAAGGCCTTATACTGGGTGCTACCATCTACCTTATAGGCTTTGATGGGACTGCGGTAATCACCAGAGCTGTGGCCGCAGACAATGGGCTAACGGCCGGCACTGACAACCTTATGCCATTCAATATTGTGATTCCAACCAGCGAGATAACCCAGCCAATCACATCCATCAAACTGGAGATAGTGACCTCCAAAAGTGGTGGTCAGGCGGGGGACCAGATGTCATGGTCAGCAAGTGGGAGCCTAGCAGTGACGATCCACGGTGGCAACTATCCAGGGGCCCTCCGTCCCGTCACACTAGTAGCCTACGAAAGAGTGGCAACAGGATCTGTCGTTACGGTCGCCGGGGTGAGCAACTTCGAGCTGATCCCAAATCCTGAACTAGCAAAGAACCTGGTCACAGAATACGGCCGATTTGACCCAGGAGCCATGAACTACACAAAATTGATACTGAGTGAGAGGGACCGTCTTGGCATCAAGACCGTATGGCCAACAAGGGAGTACACTGACTTTCGCGAGTACTTCATGGAGGTGGCCGACCTCAACTCTCCCCTGAAGATTGCAGGAGCATTTGGCTTCAAAGACATAATCCGGGCCATAAGGAGGATAGCTGTGCCGGTGGTCTCTACATTGTTCCCACCCGCCGCTCCCCTAGCCCATGCAATTGGGGAAGGTGTAGACTACCTGCTGGGCGATGAGGCACAGGCTGCTTCAGGAACTGCTCGAGCCGCGTCAGGAAAAGCAAGAGCTGCCTCAGGCCGCATAAGGCAGCTAACTCTCGCCGCCGACAAGGGGTACGAGGTAGTCGCGAATCTGTTTCAGGTGCCCCAGAATCCTGTAGTCGACGGGATTCTCGCTTCACCTGGGATACTCCGCGGTGCACACAACCTCGACTGCGTGTTGAGAGAGGGTGCCACGCTATTCCCTGTGGTCATCACGACAGTGGAAGATGCCATGACACCCAAAGCATTGAACAGCAAAATGTTTGCTGTCATTGAAGGCGTGCGAGAAGATCTACAACCTCCATCTCAAAGAGGATCCTTCATACGAACTCTCTCCGCACATAGAGTCTATGGATATGCTCCAGATGGGGTACTTCCACTGGAGACTGGGAGAGATTACACCGTGGTCCCAATAGATGATGTCTGGGATGACAGCATTATGCTGTCCAAAGACCCCATACCTCCTATTGTGGGAAACAGTAGAAACCTAGACATAGCTTACATGGATGTGTTTCGACCCAAAGTCCCCATCCATGTGGCCATGACGGGAGCCCTCAATGCCTATGGCGAGATTGAGAACGTGAGCTTTAGAAGCACCAAGCTCGCCACTGCACACCGACTTGGCCTCAAGTTGGCTGGTCCCGGTGCATTTGACGTGAACACCGGGTCCAACTGGGCAACGTTCATCAAACGTTTCCCTCACAATCCACGAGACTGGGACAGGCTCCCTTACCTCAACCTTCCATACCTTCCACCCAATGCAGGACGCCAGTACGACCTGGCCATGGCTGCTTCAGAGTTCAAAGAGACCCCCGAACTCGAGAGCGCCGTCAGAGCCATGGAAGCAGAAGCCAACGTGGACCCACTGTTCCAATCTGCACTCAGCGTGTTCATGTGGCTGGAAGAGAATGGGATTGTGACTGATATGGCCAACTTCGCACTCAGCGACCCGAATGCCCATCGGATGCGCAATTTTCTCGCAAACGCACCACAAGCAGGCAGCAAGTCGCAAAGAGCCAAGTACGGGACAGCAGGCTACGGAGTGGAGGCCCGGGGCCCCACTCCAGAGGAAGCACAGAGGGAAAAAGACACACGGATCTCAAAGAAGATGGAGACTATGGGCATCTACTTTGCAACACCAGAATGGGTAGCACTCAATGGGCACCGGGGGTCAAGCCCCGGCCAGCTAAAGTACTGGCAGAACACACGAGAAATACCTGATCCAAACGAGGACTACCTAGACTACGTGCATGCAGAGAAGAGCCGGTTGGCATCAGAAGAACAAATCCTAAGGGCAGCCACGTCGATCTACGGGGCTCCAGGACAGGCTGAGCCACCCCAGGCCTTCATAGACGAAGTCGCCAAAGTCTATGAAATCAACCATGGGCGTGGCCCCAACCAAGAACAGATGAAAGATCTGCTCTTGACTGCGATGGAGATGAAGCATCGCAATCCCAGGCGGGCTCCACCAAAGCCCAAGCCAAAACCCAATGTTCCAACACAGAGACCCCCTGGTCGGCTGGGCCGCTGGATCAGGGCTGTCTCTGATGAGGACCTTGAGTGA

SEQ ID NO: 34 - protein, 469 aa, Artificial
  Infectious bursal disease virus viral protein 2, viral protein 4 and viral protein 3 of segment A (VP2-VP4-VP3), amino acid sequence
MTNLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQAGGVTITLFSANIDAITSLSIGGELVFQTSVQGLILGATIYLIGFDGTAVITRAVAANNGLTAGTDNLMPFNIVIPTSEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRALRRIAVPVVSTLFPPAAPL

SEQ ID NO: 35 - DNA, 1707 bp, Artificial
  Influenza A virus (A/chicken/Israel/215/2007(H9N2)) segment 4 hemagglutinin (HA); Reference GenBank FJ464716.1, DNA sequence
atggaaataatatcactgatgactatactactagtagtaacaacaagcaatgcagataaaatatgcattggccaccagtcaacaaattccacagaaactgtggatacactaacagaaactaatgttcctgtgacacatgccaaagaattgctccacacagagcacaatggaatgctgtgtgcaacaaatctgggaaatcccctcatcctagacacatgcactatcgaaggacttatctatggtaacccttcttgtgacatgttgttggggggaagggaatggtcctacatcgttgaaagaccatcagcggtaaatggaacatgttaccctgggaatgtggaaaacttagaggaactcagaacactttttagttcctctagttcctatcaaagaatccaaatattcccagacacaatctggaatgtgacttacaatggaacaagcaaatcatgttcaaattcattctacaggaatatgagatggctaactcaaaagaacggggtttatcctgttcaagacgcccaatacacaaataatcggggaaaggacattcttttcgtgtggggcatacatcatccacccaccgatactgcacagacgaatttgtacacaagaaccgacacaacaacaagcgtaacaacagaaaatttagataggaccttcaaaccattgatagggccaaggccccttgtcaatggtctgattggaagaattaattattattggtcggtactaaaaccaggccagacattgcgagtaagatccaatgggaatctaattgctccatggtttggacacgttctctcaggagagagccatgggagaatcctgaaaactgatttaaacagtggtaattgtgtagtgcaatgtcagactgaaaaaggtggcctaaacagtacattgcctttccacaatatcagtaaatatgcatttgggaattgccccaaatatattggagtcaagagtctcaaactggcaatcggtctgagaaacgtgcctgccaggtcaagtagaggactatttggagccatagctggattcatagaaggaggttggccagggctagtcgccggttggtatggtttccagcattcaaatgatcaaggggttggtatggctgcagatagggattcaactcaaaaggcagttgacaaaataacatccaaggtgaataatatagtcgacaagatgaacaagcaatatgaaataattgatcatgaattcagtgaggttgaaactagactcaatatgatcaataacaagattgatgaccaaatacaagatgtatgggcatataatgcagagttgctagtactacttgagaaccagaaaacactcgatgagcatgacgcaaacgtgaacaacctatataacaaggtgaaaagggccttgggctccaatgcgatggaagatgggaagggctgtttcgagctataccacaaatgtgatgaccaatgcatggaaactattcggaacgggacctataataggagaaagtacaaagaggaatcaagactagaaaggcagaaaatagagggagtcaaactggaatctgaggggatttacaaaatacttaccatttattcgactgtcgcctcatctcttgtacttgcaatggggtttgctgccttcttattctgggccatgtccaatggatcatgcaggtgcaacatctgtatataattagcaaaaacacccttgtttcta

SEQ ID NO: 36 - protein, 560 aa, Artificial
  Influenza A virus (A/chicken/Israel/215/2007(H9N2)) segment 4 hemagglutinin (HA); Reference GenBank FJ464716.1, Amino acid sequence
MEIISLMTILLVVTTSNADKICIGHQSTNSTETVDTLTETNVPVTHAKELLHTEHNGMLCATNLGNPLILDTCTIEGLIYGNPSCDMLLGGREWSYIVERPSAVNGTCYPGNVENLEELRTLFSSSSSYQRIQIFPDTIWNVTYNGTSKSCSNSFYRNMRWLTQKNGVYPVQDAQYTNNRGKDILFVWGIHHPPTDTAQTNLYTRTDTTTSVTTENLDRTFKPLIGPRPLVNGLIGRINYYWSVLKPGQTLRVRSNGNLIAPWFGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQKIEGVKLESEGIYKILTIYSTVASSLVLAMGFAAFLFWAMSNGSCRCNICI

SEQ ID NO: 37 - DNA, 1617 bp, Artificial
  Avian infectious bronchitis virus isolate variant 1 S1 spike glycoprotein gene, Reference GenBank AF093795.1; DNA sequence
atgttgggcaaaccgcttttactagtgactctttggtatgcactatgtagtgctttgctctatgatagtagtacttacgtttactactaccaaagtgcttttaggcctagttcaggttggcacatacatgggggtgcttatgcagtagatagggtttttaatgaaaccaacaatgcaggcagtgtatctgattgcactgctggtactttttatgaaagccataatatttctgctgtttctgtagccatgacagcaccacataatggtatgtcttggtcagtttcacaattttgtacagctcattgtaacttctcagactttacagtgttcgttacgcattgttttaaaaatcaacctggtagttgtcccttgacaggtatgattcctcagaatcatattcgtatttctgctatgagacaaggaactttgttttataacttaacagttagtgtgtctaaatatcctagatttaaatcgcttcaatgtgttagcaattctacatctgtctatgtaaatggtgatcttgttttcacttctaatgaaacttcttacattacgggtgcaggcgtttattttaaaagtggtgggcctgtaacttataaagttatgaaagaagttaaagccctagcctactttattaatggtaccgcacaagaggttattttatgtgataactcacctagaggtttgcttgcatgtcagtataatactggtaatttttcagatggattctacccttttactaatcattctttagttaaggataggtttattgtatatcgagaaagtggcactaacactactttaaagttaactaatttcagttttactaatgtaagtaatgctcctcctaattcaggtggcgttgatactttccaattatatcaaacacatactgctcaggatggttattataattttaatttatcatttctgagtagttttgtgtataaaccatctgattttatgtatgggtcataccacccacattgtaattttagaccagaaaatattaataatggcttatggtttaattcattatctgtgtcacttacttacggacccattcaaggtggttgtaagcaatctgtttttaataatagagcaacttgttgctatgcttattcttatcaagggcctagtttatgtaagggtgtttatagaggggagctaatgcaatactttgaatgtggacttctagtttacgtaactaagagtgatggctctcgtatacaaactagaagtgaaccacttgtgttaactcaatataattataacaacattactttaaacaagtgtgttgagtataatatatatggtagagttggtcaaggtcttattactaatgtaactgaagcaactgctaattatagttatctagcagatggtggtttagctattttagatacctcaggagccatagacatatttgttgttcaaggtgcacatggtcttaattattataaggttaatccctgtgaggatgttaaccaacagtttgtagtgtcaggtggcaacttagttggcattcttacatctcataatgaaacaggttctgaatctattgagaaccagttttacatcaaactcactaacggaacacgtcgctctagacgt

SEQ ID NO: 38 - protein, 539 aa, Artificial
  Avian infectious bronchitis virus isolate variant 1 S1 spike glycoprotein gene, Reference GenBank AF093795.1; Amino acid sequence
MLGKPLLLVTLWYALCSALLYDSSTYVYYYQSAFRPSSGWHIHGGAYAVDRVFNETNNAGSVSDCTAGTFYESHNISAVSVAMTAPHNGMSWSVSQFCTAHCNFSDFTVFVTHCFKNQPGSCPLTGMIPQNHIRISAMRQGTLFYNLTVSVSKYPRFKSLQCVSNSTSVYVNGDLVFTSNETSYITGAGVYFKSGGPVTYKVMKEVKALAYFINGTAQEVILCDNSPRGLLACQYNTGNFSDGFYPFTNHSLVKDRFIVYRESGTNTTLKLTNFSFTNVSNAPPNSGGVDTFQLYQTHTAQDGYYNFNLSFLSSFVYKPSDFMYGSYHPHCNFRPENINNGLWFNSLSVSLTYGPIQGGCKQSVFNNRATCCYAYSYQGPSLCKGVYRGELMQYFECGLLVYVTKSDGSRIQTRSEPLVLTQYNYNNITLNKCVEYNIYGRVGQGLITNVTEATANYSYLADGGLAILDTSGAIDIFVVQGAHGLNYYKVNPCEDVNQQFVVSGGNLVGILTSHNETGSESIENQFYIKLTNGTRRSRR

SEQ ID NO: 39 - DNA, 1754 bp, Artificial
  Eimeria maxima 56 kDa gametocyte antigen (gam56) Reference GenBank AY129951.2; DNA sequence
agcagaacatagggagttcatctgttccttcttttcatcatttattcctcgtttctcaccgttttattttttttgtgtaaccctctccgctgttgagtcccaatgacccgcctcggcctcgctgctgtcgcgctggctctcgccgtgggcccttccatggcagtgcccagcaccactcctgttgagaaccaggttcacccttacagcgagatgagtacctaccaggaggggagtgccccgggggctccggaggacaccaccaccaccactacgtcgtcccctgtttccgatggagccgagcagtggcttgagagctttgttcgtgctgtgcagcgccagctgcagcttcaggaccaaatgatgcgtcagctcatgagggacattcaggagtacctgagcactgcgttcaactgggcagagaaccagtctactgcctacacccgtgttaccgagatgatggacatgatctccaacagaatgaacgctgccatggacagctcaaacgaactcatgaccactagcgacaccacagaccccgagaccctccgccgtgcaactcgcaagtacatgaaggaggttcgcgttcaggacgtcctggtagatgctctctgggcctctctccgcggtgtacagacagctgcctggatgaatggagtgaccgctattgagaaggaggagacgactcccatggctagccgcgctgctgaggagttcctccaccgcatgtaccataacctgagggcagcaggtatgtctgaagaagatgttgccaagttcatccctagagccgagtacaacccctccgagcagtcaagaaatatgggcagaaagggcaggagcttctactacggcggctatcccagctactacaactccccctactacagctacagcagctaccccagctactacaactacagctacccgtcatacagctacagcagctaccccagctactaccgctacagcagctacccctactacaactacagctatcccagctactacaactacggcagctacccctactacagttatagcagctaccccagctggtactggcgccgtctccgctctttggcaacagcaacttgcccagactgccctcctctcaccactcccagcatgatcccaactccccccccaatgatgaacatgatgaacaccccaccccccatggcaaacatgatgaccagcatgatgatgaacactcccatggttcctcctccccgcaccctcggaactgaagccatgagcctcggcttggcccccatcggtatcaccggcgcccccatgacaggtttcggtgttcctcctgagttcggtccctttggagccgaaggtatcggcctccccaccgatgccctcggcagcacccccgaaatgacaccattcgacccaactaccccctacagaactctcgcccccatggacctcccccccatcccccctcctgtcttccctgaaacccctatgaggccacctactcccttcggcttcggacctgcacctgttcctcccatgcccttctaaacgacctaccatccctcaatccatagctcacatttcgtagcctcaaaacagttttttgttcatttcacttccaggactcatgctgcgacatttgcattcgtacctcgaaaccgtcaacctcaaaccccaaaccattctgtgacctcccctcgcaaacgcggaaggcggaacattttttctgaagtatattactacgttaaaaaaaaaaaaaaaaaaaaaaa

SEQ ID NO: 40 - protein, 476 aa, Artificial
  Eimeria maxima 56 kDa gametocyte antigen (gam56) Reference GenBank AY129951.2; Amino acid sequence
MTRLGLAAVALALAVGPSMAVPSTTPVENQVHPYSEMSTYQEGSAPGAPEDTTTTTTSSPVSDGAEQWLESFVRAVQRQLQLQDQMMRQLMRDIQEYLSTAFNWAENQSTAYTRVTEMMDMISNRMNAAMDSSNELMTTSDTTDPETLRRATRKYMKEVRVQDVLVDALWASLRGVQTAAWMNGVTAIEKEETTPMASRAAEEFLHRMYHNLRAAGMSEEDVAKFIPRAEYNPSEQSRNMGRKGRSFYYGGYPSYYNSPYYSYSSYPSYYNYSYPSYSYSSYPSYYRYSSYPYYNYSYPSYYNYGSYPYYSYSSYPSWYWRRLRSLATATCPDCPPLTTPSMIPTPPPMMNMMNTPPPMANMMTSMMMNTPMVPPPRTLGTEAMSLGLAPIGITGAPMTGFGVPPEFGPFGAEGIGLPTDALGSTPEMTPFDPTTPYRTLAPMDLPPIPPPVFPETPMRPPTPFGFGPAPVPPMPF

SEQ ID NO: 41 - DNA, 21 bp, Artificial
  DNA primer
atcaggacagcaaagacagca

SEQ ID NO: 42 - DNA, 21 bp, Artificial
  DNA primer
gcctcagtcgtgtgcttcttg

(19) United States
(12) Patent Application Publication
(10) Pub. No.: US 2016/0031991 A1
(43) Pub. Date: Feb. 4, 2016

(54) TARGETING VACCINES FOR VETERINARY USE

(71) Applicant: VACCIBODY AS, Olso (NO)

(72) Inventors: Ole Henrik BREKKE, Oslo (NO); Mona Mari LINDEBERG, Oslo (NO); Agnete Brunsvik FREDRIKSEN, Rællingen (NO)

(73) Assignee: VACCIBODY AS, Oslo (NO)

(21) Appl. No.: 14/776,001

(22) PCT Filed: Mar. 13, 2014
(86) PCT No.: PCT/EP2014/054961

§ 371 Date: Sep. 14, 2015

Related U.S. Application Data
(60) Provisional application No. 61/788,102, filed on Mar. 15, 2013.

(51) Int. Cl. C07K 16/28; C12N 7/00; A61K 39/395; C07K 14/005

(52) U.S. Cl. CPC C07K 16/2833; C07K 14/005; C12N 7/00; A61K 39/3955; C07K 2317/524; C07K 2317/53; C12N 2710/16631; C07K 2317/622; C07K 2319/00

(57) ABSTRACT

The present invention relates to therapeutic compounds, such as vaccines against avian diseases and in particular to DNA vaccines. The invention further relates to protein construct encoding homodimeric peptides, which peptides may be released from a DNA vaccine or used separately. Further described are pharmaceutical formulations, host cells and methods for producing the vaccines, as well as methods for the treatment or prevention of various diseases in animals, such as avians, such as cancers and infectious diseases.

### FIELD OF THE INVENTION

The present invention relates to therapeutic compounds, such as vaccines against avian diseases and in particular to DNA vaccines. The invention further relates to DNA construct encoding homodimeric peptidesas well as vector vaccines. The encoded peptides may be expressed from the DNA vaccine construct directly within the host receiving the vaccine or be produced and used as recombinant peptides separately. Further described are pharmaceutical formulations, host cells and methods for producing the vaccines, as well as methods for the treatment or prevention of various diseases in animals, such as avians, such as cancers and infectious diseases.

### BACKGROUND OF THE INVENTION

The global veterinary vaccines market is expected to post substantial growth in ensuing years. Growing demand for the vaccines from Asia, Latin America and Eastern European countries and increased vulnerability of animals to the diseases is steering the demand for veterinary vaccines. Rapidly changing patterns of the diseases among the animals and increased development of resistance to the currently used antimicrobials is compelling the manufacturers to invest heavily in new product developments. Adoption of novel animal husbandry techniques and different farming conditions are attributed as the major factors for emergence of newer diseases. Growing awareness on animal health and benefits of early detection and preventive medicines will drive the demand for veterinary vaccines. Technology innovations, in particular DNA-vaccines, replicating and non-replicating vector vaccines, and introduction of new products that are capable of ensuring greater production and immune responses than traditional vaccines also offers good prospects for the future of veterinary vaccines market.

The global veterinary vaccines market ($4.23 Bn) accounted for around 20% of the total vaccines market ($29.71 Bn) in 2010. It is expected to grow with a CAGR of 5.80% to reach the market size of $5.6 Bn by 2015. United States represents the largest market for veterinary vaccines worldwide, with the market share of 46% and market size of $1.94 Bn in 2010.

The market segment for livestock vaccines accounts for approximately $2 Bn and is composed of vaccines for cattle, pigs, sheep, poultry and within aquaculture. The efforts in vaccine development within the livestock segment are driven by the need for vaccines obtaining better, safer and more effective responses. Additionally, ways to easily administer vaccines in herds, such as for mass vaccination of chickens, both with respect to the number of doses needed to be given and methods of facile administration, is a challenging demand.

Chickens express many of the regulatory proteins that mammals do. Much effort is on-going to augment immune responses or even alter a bird's capacity to respond to vaccines. Avian dendritic cells are now being characterized and the research suggests that these cells, like their mammalian counterparts, are the key antigen presenting cell in the initiation of a robust immune response. Targeting avian dendritic cells with vaccines therefore should be an attractive approach for obtaining effective immune responses for novel chicken vaccines.

There is a need for improved vaccines against poultry diseases including avian coccidiosis, necrotic enteritis, avian encephalomyelitis, avian infectious bronchitis, avian infectious bursal disease, avian reovirus, chicken anaemia virus, duck virus enteritis, egg drop syndrome 1976, erysipelas, infectious laryngotracheitis, Marek's disease, Newcastle disease, pasteurellosis, post-natal colibacillosis, salmonellosis, swollen head syndrome, turkey haemorrhagic enteritis, turkey rhinotracheitis and avian influenza.

### OBJECT OF THE INVENTION

Vaccibodies (International patent application number PCT/EP2012/076404, WO2011161244, and WO2004076489) are vaccines, DNA constructs that harbours the ability to express protein molecules targeting antigen presenting cells (APC), such as dendritic cells, in vivo and in vitro by being directed towards specific surface receptors on the APCs. Vaccibody can be delivered both as a DNA vaccine, a vector vaccine, or the encoded protein subunit vaccine. Alternatively, Vaccibody constructs within the present invention may be used to activate APC in vitro, and then the activated APC may be used for vaccination.

The invention describes Vaccibody vaccines based on the format described in FIG. 1, which are composed of a targeting protein module (herein also referred to as a targeting unit) binding to receptors expressed on the cell surface of APCs. In this specific embodiment, the targeting unit is composed of a scFv fragment that is recognising and binding chicken MHC class II molecules on avian cells. A dimerization protein module composed of hinge regions, hinge-like regions and constant heavy chain domains from human, rodent, bovine or avian immunoglobulins, which connects two vaccibody monomers generating a homodimer molecule. And the antigenic unit (vaccine module) that can be any protein unit such as a domain, short segment or a peptide or combinations of different protein units, derived from a pathogen or cancer related tissue.

The invention describes the novel vaccine format that is designed for obtaining significant better and more effective vaccines for veterinary purposes, which can be delivered as a DNA vaccine, vector vaccine, or protein vaccines by different administration methods, such as in ovo, in the drinking water, as aerosol spray, delivered by jet injectors, as needle injection or as viral vector delivery methods.

It is an object of embodiments of the invention to provide specific and highly effective therapeutic compounds, such as DNA vaccines against diseases and conditions in animals, such as in avian species.

### SUMMARY OF THE INVENTION

It has been found by the present inventor(s) that a scFv fragment specifically binding avian MHC class II molecule, such as chicken MHC class II molecule, on avian cells may be used as a highly efficient targeting unit in the design of vaccines in a vaccibody structure.

So, in a first aspect the present invention relates to a homodimeric protein of two identical amino acid chains, each amino acid chain comprising (1) optionally a signal peptide, (2) a targeting unit, (3) a dimerization motif, and (4) an antigenic unit, said targeting unit being a scFv fragment specifically binding avian MHC class II molecule, such as chicken MHC class II molecule, on avian cells.

It is to be understood that the constructs according to the present invention only require a signal peptide in a form where it is to be exported out of a cell producing such construct. Accordingly, a nucleic acid construct usually have to contain a sequence encoding the signal peptide to have a final protein exported from the cell producing such protein. However, if produced and administered as a recombinant protein, a signal peptide may not be required. In some embodiments, the homodimeric protein of two identical amino acid chains does not contain a signal peptide.

In a second aspect, the present invention relates to an amino acid chain comprising (1) an optional signal peptide, (2) a targeting unit, (3) a dimerization motif, and (4) an antigenic unit, said targeting unit being a scFv fragment specifically binding chicken MHC class II molecule on avian cells, which amino acid chain is able to form a homodimeric protein according to the present invention.

In a third aspect the present invention relates to a nucleic acid molecule, such as a DNA, encoding an amino acid chain comprising (1) an optional signal peptide, (2) a targeting unit, (3) a dimerization motif, and (4) an antigenic unit, said targeting unit being a scFv fragment specifically binding chicken MHC class II molecule on avian cells, which amino acid chain is able to form a homodimeric protein according to the present invention.

In a further aspect, the present invention relates to an amino acid chain comprising (1) an optional signal peptide, (2) a targeting unit, (3) an optional dimerization motif, and (4) an antigenic unit, said targeting unit being a scFv fragment specifically binding chicken MHC class II molecule on avian cells.

In a further aspect the present invention relates to a vector, such as a viral vector or a plasmid vector, such as one optimized for avians comprising the nucleic acid molecule according to the invention. In some embodiments the vector is able to express the nucleic acid molecule as a functional protein in avian cells

In a further aspect the present invention relates to a homodimeric protein according to the invention, or an amino acid chain according to the invention, or the nucleic acid molecule according to the invention or a vector according to the invention for use as a medicament.

In a further aspect the present invention relates to a pharmaceutical composition comprising a homodimeric protein according to the invention, or an amino acid chain according to the invention, or the nucleic acid molecule according to the invention, or a vector according to the invention.

In a further aspect the present invention relates to a host cell comprising the nucleic acid molecule according to the invention, or a vector according to the invention.

In a further aspect the present invention relates to a method for preparing a homodimeric protein according to the invention, or an amino acid chain of the invention, the method comprising a) transfecting the nucleic acid molecule according to the invention or a vector according to the invention into a cell population, such as eukaryotic, bacterial or yeast cells; b) culturing the cell population; c) collecting and purifying the homodimeric protein, or amino acid chain expressed from the cell population.

In a further aspect the present invention relates to a method for preparing a vaccine, such as a DNA vaccine, comprising an immunologically effective amount of a nucleic acid molecule according to the invention or a vector according to the invention, the method comprising a) preparing a nucleic acid molecule according to the invention, or a vector according to the invention; b) dissolving the nucleic acid molecule or vector obtained under step a) in a pharmaceutically acceptable carrier, diluent, or buffer.

In a further aspect the present invention relates to a method for preparing a cell, such as an antigen presenting cell vaccine or cell line producing the homodimeric protein according to the invention, or the amino acid chain according to the invention, the method comprising; a) preparing a nucleic acid molecule according to the invention, or vector according to the invention; b) activating in vitro the cells, such as antigen presenting cells with an immunologically effective amount of a nucleic acid molecule or vector prepared under step a); and c) preparing the cells, such as antigen presenting cells obtained under step b) in a suitable diluent, such as a pharmaceutically acceptable carrier, diluent, or buffer.

In a further aspect the present invention relates to a vaccine against a disease or condition in an animal, such as a cancer or an infectious disease caused by a virus, bacteria, protozoa, or other infectious agent, the vaccine comprising an immunologically effective amount of a homodimeric protein according to the invention, or an amino acid chain according to the invention, or nucleic acid molecule, such as a DNA, according to the invention, or vector according to the invention, wherein said vaccine is able to trigger both a T-cell- and/or B-cell immune response.

In a further aspect the present invention relates to a method of treating or preventing a disease or condition in an animal, such as a cancer or an infectious disease caused by a virus, bacteria or other infectious agent, the method comprising administering to said animal in need thereof, a homodimeric protein according to the invention, or an amino acid chain according to the invention, or the nucleic acid molecule, such as a DNA, according to the invention, or vector according to the invention.

### LEGENDS TO THE FIGURE

FIG. 1: The vaccibody structure

FIG. 2: The curve shows effective protein production and secretion of 2G11-vaccibody (absorbance at 450 nm) into the supernatant of transiently transfected HEK293E cells. OD levels observed in sandwich ELISA using anti-human IgG (MCA878) and anti-His antibodies performed on dilutions from supernatant of three independent transient transfections with 2G11 vaccibody constructs (ScFv2G11lhgD-His) and one negative control (EB=Elisa Buffer). MCA878 recognizes the dimerization unit from human IgG3 and anti-His antibody recognized the 6×-His tag included C-terminal to the HSV-2 gD antigenic unit.

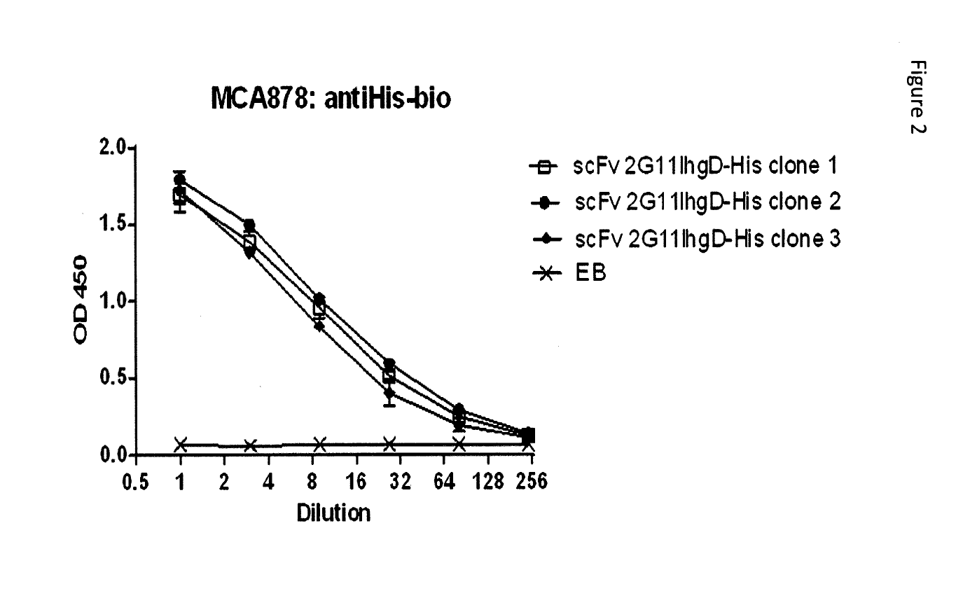

FIG. 3: Flowcytometry analysis on binding to CD45+ chicken PBMC. Isolated chicken PBMC were stained with 2G11 vaccibody and negative controls (Staining buffer, non-targeted aNIP control and isotype control). Binding analysis were performed on gated CD45+ cells using overlaying histograms.

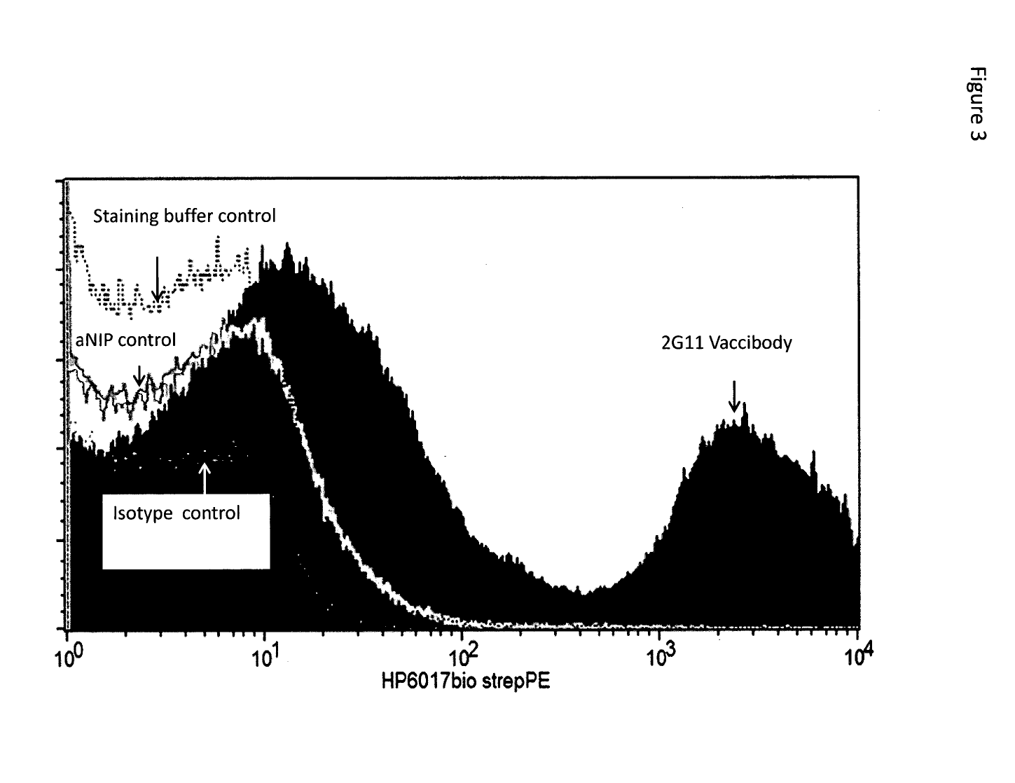

FIG. 4: Flowcytometry analysis on binding to CD45+ chicken PBMC. Isolated chicken PBMC were stained with 2G11 vaccibody and positive (2G11 mAb-AH diagnostics) and negative controls (non-targeted aNIP control and isotype control). Binding analysis were performed on gated CD45+ cells using overlaying histograms. Purple filled: 2G11 vaccibody, Green: 2G11 mAb (positive control), Pink: NlhCkCk (non-targeting vaccibody, irrelevant targeting unit), Blue line: 2G11 vaccibody isotype control

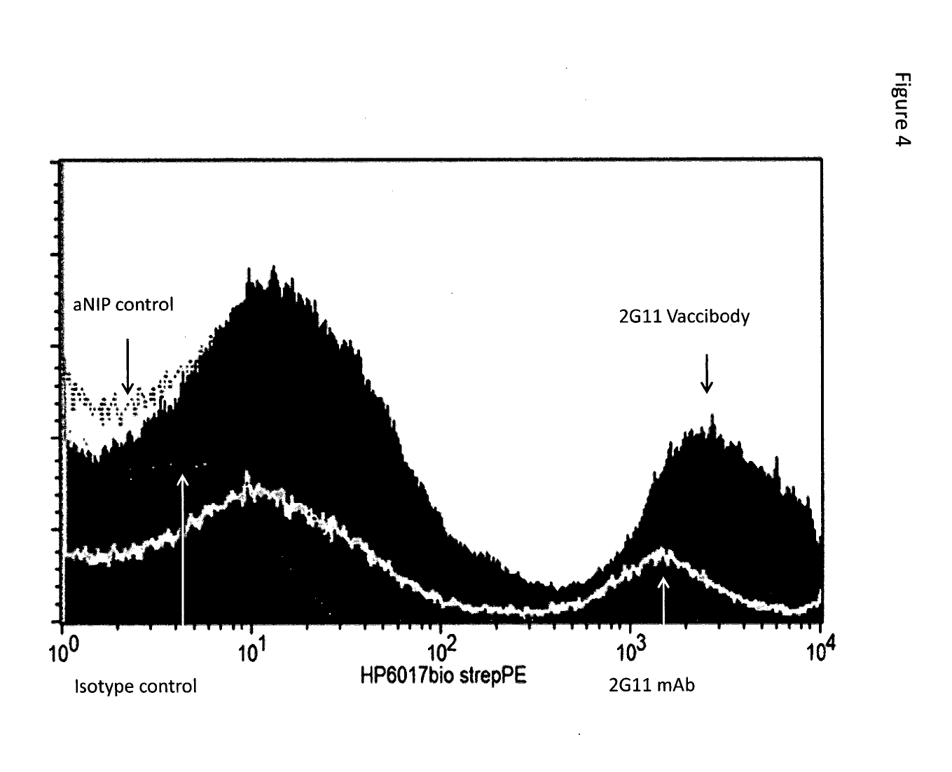

FIG. 5: Flowcytometry analysis on binding to CD45+ chicken PBMC. Isolated chicken PBMC were stained with the 2G11 mAb (AH Diagnostics) and negative controls (Staining buffer and isotype control). Binding analysis were performed on gated CD45+ cells using overlaying histograms. Purple filled: 2G11 mAb (positive control), Green: Staining Buffer, Pink: 2G11 mAb isotype control.

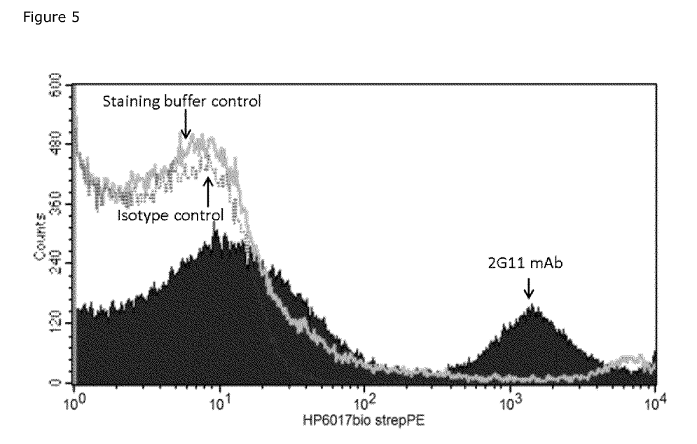

### DETAILED DISCLOSURE OF THE INVENTION

The DNA and protein constructs and DNA vaccine technology described herein by the inventors of the present invention (also referred to as “vaccibody” molecules/vaccines/constructs) represents a novel vaccine strategy to induce strong and specific immune responses for both infectious diseases, such as avian infectious diseases and cancer. The vaccine described herein may be administered as a DNA vaccine by intradermal or intramuscular or in ovo injection, or via the respiratory/mucosal/GI-tract. This results in the uptake of the DNA-construct encoding the vaccibody-vaccine in cells at the site of administration, leading to in vivo production of the vaccibody-protein molecule. In alternative aspects, the constructs according to the present invention are produced as recombinant protein vaccines and administered by similar means as the DNA vaccine.

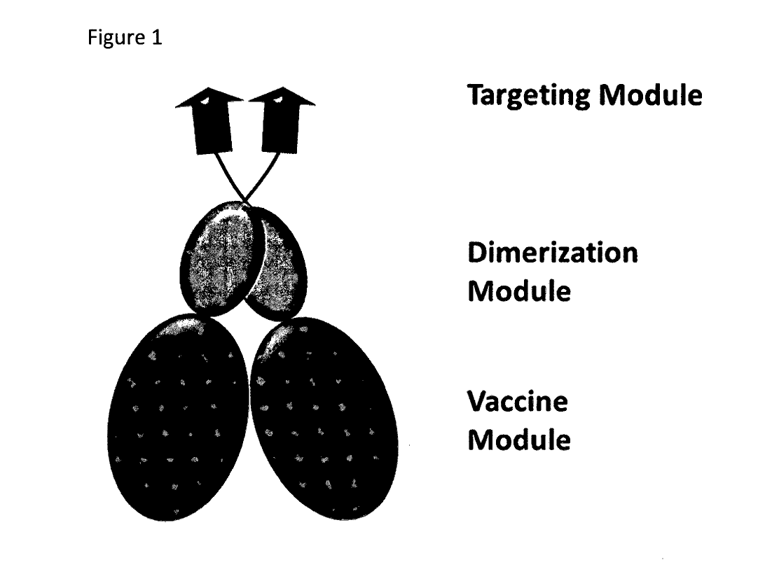

The vaccibody molecule described herein is a homodimer consisting of three modules; targeting module (or unit), dimerization module (or motif) and the antigenic unit (vaccine module) (FIG. 1). Genes encoding the three modules are genetically engineered to be expressed as one gene translated to one protein. When expressed in vivo, the vaccibody molecule targets MHC class II molecules on antigen-presenting cells which may result in enhanced vaccine potency compared to identical, non-targeted antigens.

The invention describes several variants of Vaccibody vaccines, all based on the overall format described in FIG. 1. The vaccibody vaccines is composed of a targeting unit encoding a scFv fragment specifically binding chicken MHC class II molecule on avian cells.

The dimerization module genes may encode hinge regions and constant heavy chains, such as domains from avian IgY which connects two vaccibody monomers generating a homodimer molecule. Genes encoding the antigenic unit (vaccine module) for the current strategy may be for any protein unit such as a domain, short segment or a peptide or combinations of different protein units, derived from a pathogen or cancer related tissue. Once the DNA vaccine is administered in vivo, cells receiving the vaccine construct will express the vaccibody protein molecule. The in vivo produced vaccibody vaccines target MHC class II molecule expressed on the surface of APCs. The binding of the vaccibody molecule to its cognate receptors leads to internalization of the complex in the APC, degradation of the proteins into small peptides that are loaded onto MHC molecules and presented to CD4⁺ and CD8⁺ T cells to induce a specific immune response to the antigenic unit. Once stimulated and with help from activated CD4⁺ T cells, CD8⁺ T cells will target and kill antigen expressing cells. Such enhanced immune responses to a vaccine with a “built-in” adjuvant effect may potentially overcome tumor-escape by breaking immunological tolerance and efficiently kill malignant cells, or enhancing the immune response to pathogens. The targeting unit by the scFv fragment specifically binding chicken MHC class II molecule may be connected through a dimerization motif, such as a hinge region/shortened CH2 domain, to an antigenic unit, wherein the later is in either the COOH-terminal or the NH2-terminal end. The present invention not only relates to a DNA sequence coding for this recombinant protein, but also to expression vectors comprising these DNA sequences, cell lines comprising said expression vectors, to treatment of mammals and avians preferentially by immunization by means of Vaccibody DNA, Vaccibody RNA, or Vaccibody protein, and finally to pharmaceuticals and a kit comprising the said molecules.

As used herein the terms “treatment” or “treating” refers to preventing, alleviating, managing, curing or reducing one or more symptoms or clinically relevant manifestations of a disease or disorder, unless contradicted by context. For example, “treatment” of a subject, such as an avian population in whom no symptoms or clinically relevant manifestations of a disease or disorder have been identified is preventive or prophylactic therapy, whereas “treatment” of subjects in which symptoms or clinically relevant manifestations of a disease or disorder have been identified generally does not constitute preventive or prophylactic therapy. The dimerization motif in the proteins according to the present invention may be constructed to include a hinge region and/or immunoglobulin domains (e.g. Cγ3 domain from human IgG3 or C_{H}2 and/or C_{H}4 domain of avian IgY, or a sequence that is substantially identical to said C domain. The hinge region may be Ig, such as IgG derived and contributes to the dimerization through the formation of an interchain covalent bond(s), e.g. disulfide bridge(s). In addition, it functions as a flexible spacer between the domains allowing the two targeting units to bind simultaneously to two target receptors on APC expressed with variable distances. The immunoglobulin domains contribute to homodimerization through non-covalent interactions, e.g. hydrophobic interactions. In one embodiment the dimerization motif comprises a C_{H}3 domain, such as one derived from IgG. In one embodiment the dimerization motif comprises a C_{H}4 domain, such as one derived from IgY. These dimerization motifs may be exchanged with other multimerization moieties (e.g. from other Ig isotypes/subclasses). Preferably the dimerization motif is derived from native proteins, such as IgG, IgM or IgY molecules from relevant species, such as from an avian species, such as from chicken.

It is to be understood that the dimerization motif may have any orientation with respect to antigenic unit and targeting unit. In one embodiment the antigenic unit is in the COOH-terminal end of the dimerization motif with the targeting unit in the N-terminal end of the dimerization motif. In another embodiment the antigenic unit is in the N-terminal end of the dimerization motif with the targeting unit in the COOH-terminal end of the dimerization motif.

International application WO 2004/076489, PCT/EP2012/076404, and WO2011161244, which is hereby incorporated by reference discloses nucleic acid sequences and vectors, which may be used according to the present invention.

The proteins according to the present invention include an antigenic unit, as well as immunogenic fragments or variants thereof. The antigenic sequence should be of sufficient length. The minimal length of such antigenic unit may be around 9 amino acids. Accordingly in some embodiments, the antigenic unit comprises an amino acid sequence of at least 9 amino acids corresponding to at least about 27 nucleotides in a nucleic acids sequence encoding such antigenic unit.

Immunization by means of Vaccibody protein, Vaccibody DNA, or Vaccibody RNA, the latter two executed e.g. by intramuscular or intradermal injection with or without a following electroporation, are all feasible methods according to the present invention.

As discussed above, the present invention relates to a vaccine composition that may be used against any cancer or infectious diseases, the vaccine composition comprising an immunologically effective amount of the nucleic acid encoding the molecule of the invention or degenerate variants thereof. The vaccine may be able to trigger both a T-cell- and B-cell immune response. The present invention also relates to a kit comprising Vaccibody DNA, RNA, or protein for diagnostic, medical or scientific purposes.

The invention further relates to a method of preparing the recombinant molecule of the invention comprising, transfecting the vector comprising the molecule of the invention into a cell population; culturing the cell population; collecting recombinant protein expressed from the cell population; and purifying the expressed protein.

The above described nucleotide sequences may be inserted into a vector suited for gene therapy, e.g. under the control of a specific promoter, and introduced into the cells. It is to be understood that the term vector as used herein refers to any molecule or construct suitable for delivering the nucleotide sequences according to the present invention. In some embodiments the vector is a plasmid vector. In some embodiments the vector is a viral vector.

In some embodiments the vector comprising said nucleotide sequence is a virus (a viral vector), e.g. Lentivirus, an adenovirus, alphavirus, herpes, vaccinia virus or an adeno-associated virus, or alternatively avian vectors derived from HVT, fowl pox, newcastle or any other avian vector. In some embodiments a retroviruse is used as vector. Examples of suitable retroviruses are e.g. MoMuLV or HaMuSV. For the purpose of gene therapy, the DNA/RNA sequences according to the invention can also be transported to the target cells in the form of colloidal dispersions. They comprise e.g. liposomes or lipoplexes. Nonviral substances such as Ormosil may also be used as vectors and may deliver nucleotide sequences to specifically targeted cells in living animals.

The present invention encompasses the use of a targeting unit, an antigenic unit, as well as a dimerization motif comprising a hinge region and optionally carboxyterminal C domains and linkers, each domain having minimum degree of sequence identity or sequence homology with amino acid sequence(s) defined herein or with a polypeptide having the specific properties defined herein. The present invention encompasses, in particular, the use of peptide variants or peptide units to be used in the constructs according to the present invention having a degree of sequence identity with any one of SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40. Here, the term “variant” means an entity having a certain degree of sequence identity with the subject amino acid sequences or the subject nucleotide sequences, where the subject amino acid sequence preferably is SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40.

In one aspect, the variant or fragment amino acid sequence and/or nucleotide sequence should provide and/or encode a polypeptide which retains the functional activity and/or enhances the activity of a polypeptide of SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40.

In the present context, a variant sequence is taken to include an amino acid sequence which may be at least 70%, 75%, 80%, at least 85%, at least 90%, at least 95%, at least 96%, at least 97%, at least 98% or at least 99%, identical to the subject sequence. Typically, the variants used according to the present invention will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Sequence identity comparisons can be conducted by eye, or more usually, with the aid of readily available sequence comparison computer programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/ and which was firstly described in Altschul et al. (1990) J. Mol. Biol. 215; 403-410.

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools—sequence analysis—ClustalW2. Currently, the exact address of the ClustalW2 tool is www.ebi.ac.uk/Tools/clustalw2.

In another embodiment, it is preferred to use the program Align X in Vector NTI (Invitrogen) for performing sequence alignments. In one embodiment, Exp10 may be used with default settings:

Gap opening penalty: 10
Gap extension penalty: 0.05
Gap separation penalty range: 8
Score matrix: blosum62mt2

Thus, the present invention also encompasses the use of variants, fragments, and derivatives of any amino acid sequence of a protein, polypeptide, motif or domain as defined herein, particularly those of SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40.

The sequences, particularly those of variants, fragments, and derivatives of SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue, with an alternative residue) that may occur i.e. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

Replacements may also be made by unnatural amino acids include; alpha* and alpha-disubstituted* amino acids, N-alkyl amino acids*, lactic acid*, halide derivatives of natural amino acids such as trifluorotyrosine*, ρ-Cl-phenylalanine*, ρ-Br-phenylalanine*, ρ-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, ρ-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-conservative substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al. (1992), Horwell D C. (1995).

In one embodiment, the variant targeting unit used in the homodimeric protein according to the present invention is variant having the sequence of amino acids at least 80%, at least 85%, at least 90%, at least 95%, at least 96%, at least 97%, at least 98% or at least 99% amino acid sequence identity therewith.

In one aspect, preferably the protein or sequence used in the present invention is in a purified form. The term “purified” means that a given component is present at a high level without significant levels of other protein or sequences, such as being 99%, 95%, 90%, 85%, 80%, 75%, or 70% pure. The component is desirably the predominant active component present in a composition.

A “variant” or “variants” refers to proteins, polypeptides, units, motifs, domains or nucleic acids. The term “variant” may be used interchangeably with the term “mutant.” Variants include insertions, substitutions, transversions, truncations, and/or inversions at one or more locations in the amino acid or nucleotide sequence, respectively. The phrases “variant polypeptide”, “polypeptide”, “variant” and “variant enzyme” mean a polypeptide/protein that has an amino acid sequence that has been modified from the amino acid sequence of SEQ ID NO: 1. The variant polypeptides include a polypeptide having a certain percent, e.g., 70%, 75%, 80%, 85%, 90%, 91%, 92%, 93%, 94%, 95%, 96%, 97%, 98%, or 99%, of sequence identity with the amino acid sequence of SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40.

“Variant nucleic acids” can include sequences that are complementary to sequences that are capable of hybridizing to the nucleotide sequences presented herein. For example, a variant sequence is complementary to sequences capable of hybridizing under stringent conditions, e.g., 50° C. and 0.2×SSC (1×SSC=0.15 M NaCl, 0.015 M sodium citrate, pH 7.0), to the nucleotide sequences presented herein. More particularly, the term variant encompasses sequences that are complementary to sequences that are capable of hybridizing under highly stringent conditions, e.g., 65° C. and 0.1×SSC, to the nucleotide sequences presented herein. The melting point (Tm) of a variant nucleic acid may be about 1, 2, or 3° C. lower than the Tm of the wild-type nucleic acid. The variant nucleic acids include a polynucleotide having a certain percent, e.g., 80%, 85%, 90%, 95%, or 99%, of sequence identity with the nucleic acid encoding SEQ ID NO:7, SEQ ID NO:9, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:36, SEQ ID NO:38, or SEQ ID NO:40, encoding the monomeric protein which can form the homodimeric protein according to invention.

### Signal Peptide:

A signal peptide at the N-terminal end of the nascent polypeptide directs the molecule into the ER before transport into the Golgi complex. The signal peptide is cleaved off by signal peptidase once it has served its purpose of targeting and importing the protein to the ER. These signal peptides are generally between 15 and 30 amino acids, but can have more than 50 residues (Martoglio, B. et al., Trends in Cell Biology, 1998, Knappskog, S. et al., J Biotechnol, 2007). The native signal peptide may be replaced by signal peptides from any mammalian, avian, prokaryotic or marine origin. Commonly used signal peptides are e.g. human IL-2 and human albumin due to their natural ability to secrete large amounts of protein. The choice of signal peptide can have a considerable impact on the amount of synthesized and secreted protein.

In some embodiments the signal peptide is not derived from pLNOH2 (B1-8 variable immunoglobulin leader) disclosed in the international application with International Application No: PCT/EP2011/060628.

In some embodiments the signal peptide is not derived from an immunoglobulin gene.

In some embodiments the signal peptide is derived from classes of peptides known to effectively secrete proteins from avian cells, such as chicken IL-2 signal peptide or chicken immunoglobulin signal peptides.

### Homodimeric Protein:

The term “homodimeric protein” as used herein refers to a protein comprising two individual identical strands of amino acids, or subunits held together as a single, dimeric protein by hydrogen bonding, ionic (charged) interactions, actual covalent disulfide bonding, or some combination of these interactions.

The term “dimerization motif”, as used herein, refers to the sequence of amino acids between the antigenic unit and the targeting unit comprising the hinge region/shortened CH2 domain and the optional second domain that may contribute to the dimerization. This dimerisation motif may be of immunoglobulin origin, and optionally the hinge region/shortened CH2 domain and the second domain are connected through a linker. Accordingly the dimerization motif serves to connect the antigenic unit and the targeting unit, but also facilitates the dimerization of the two monomeric proteins into a homodimeric protein according to the invention.

It is to be understood that for some aspects of the present invention, wherein the construct only contain a single amino acid chain comprising optionally a signal peptide, a targeting unit, and an antigenic unit, then a dimerization motif may be absent from the construct.

### Targeting Unit:

The term “targeting unit” as used herein refers to a unit that delivers the protein with its antigen to mammalian or avian APC for MHC class II-restricted presentation to CD4+ T cells or for providing cross presentation to CD8+ T cells by MHC class I restriction. The targeting unit used in the constructs according to the present invention is a single chain Fv fragment specifically binding chicken MHC class II molecules on avian cells, such as one derived from the hybridoma MaD2G11.

### Antigenic Unit:

The term “antigenic unit” as used herein refers to any molecule, such as a peptide which is able to be specifically recognized by an antibody or other component of the immune system, such as a surface receptor on T-cells. Included within this definition are also immunogens that are able to induce an immune response. The terms “epitope” or “antigenic epitope” is used to refer to a distinct molecular surface, such as a molecular surface provided by a short peptide sequence within an antigenic unit. In some embodiments the antigenic unit comprises two or more antigenic epitopes. The antigenic unit used in the constructs according to the present invention may be derived from herpes simplex virus 2 glycoprotein D (gD), but any antigenic unit suitable for preventive and/or therapeutic effect for e.g. poultry diseases may be utilized This includes antigens from agents causing avian coccidiosis, avian encephalomyelitis, avian infectious bronchitis, such as S protein, S1 protein or S2 protein, avian infectious bursal disease, such as VP2 protein, avian reovirus, such as Sigma Cc protein, chicken anaemia virus, duck virus enteritis, egg drop syndrome 1976, erysipelas, infectious laryngotracheitis, Marek's disease, Newcastle disease, such as Hemagglutinin-neuraminidase fusion protein pasteurellosis, post-natal colibacillosis, salmonellosis, swollen head syndrome, turkey haemorrhagic enteritis, turkey rhinotracheitis and avian influenza, such as HA proteins, such as HA1, HA2, HA3, HA4, HA5, HA6, HA7, HA8, HA9, HA10, HA11, HA12, HA13, HA14, or HA15.

The term “hinge region” refers to a peptide sequence of the homodimeric protein that facilitates the dimerization, such as through the formation of an interchain covalent bond(s), e.g. disulfide bridge(s). The hinge region may be Ig derived, such as hinge exons h1+h4 of an Ig, such as IgG3, or equivalent units derived from avian immunoglobulin molecules, e.g. CH2 domain from IgY.

### Formulations

The DNA constructs and encoded proteins of the invention can be formulated using one or more excipients to increase stability; increase cell transfection; permit a sustained or delayed release; increase the translation of encoded protein in vivo; and/or alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with modified nucleic acid, hyaluronidase, nanoparticle mimics and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to one-half or one-third of such a dosage.

Relative amounts of the active ingredient, the pharmaceutically acceptable excipient, and/or any additional ingredients in a pharmaceutical composition in accordance with the present invention may vary, depending upon the identity, size, and/or condition of the population being treated and further depending upon the route by which the composition is to be administered. For example, the composition may comprise between 0.1% and 99% (w/w) of the active ingredient.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21.sup.st Edition, A. R. Gennaro, Lippincott, Williams & Wilkins, Baltimore, Md., 2006; incorporated herein by reference in its entirety).

### Specific Embodiments of the Invention

A stated above the present invention relates to a homodimeric protein of two identical amino acid chains, each amino acid chain comprising (1) optionally a signal peptide, (2) a targeting unit, (3) a dimerization motif, and (4) an antigenic unit, said targeting unit being a scFv fragment specifically binding avian MHC class II molecule, such as chicken MHC class II molecule, on avian cells.

In some embodiments, the targeting unit comprises an amino acid sequence having at least 80% sequence identity to the amino acid sequence of SEQ ID NO:7.

In some embodiments, the targeting unit comprises an amino acid sequence having at least 80% sequence identity to the amino acid sequence of SEQ ID NO:9.

In some embodiments, the targeting unit comprises an amino acid sequence having at least 80% sequence identity to the amino acid sequence of SEQ ID NO:11.

In some embodiments, the antigenic unit is any protein unit such as a domain, short segment or a peptide or combinations of different protein units, derived from a pathogen or cancer related tissue.

In some embodiments, the targeting unit, dimerization motif and antigenic unit in said amino acid chain are in the N-terminal to C-terminal order of targeting unit, dimerization motif and antigenic unit.

In some embodiments, the targeting unit comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the amino acid sequence of SEQ ID NO:7.

In some embodiments, the targeting unit comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the amino acid sequence of SEQ ID NO:9.

In some embodiments, the targeting unit consists of an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the amino acid sequence of SEQ ID NO:11.

In some embodiments, the dimerization motif comprises a hinge region and optionally another domain that facilitate dimerization, such as an immunoglobulin domain, optionally connected through a linker.

In some embodiments, the hinge region is Ig derived, such as a shortened CH2 domain from IgY, such as an Ig derived from avian, such as chicken.

In some embodiments, the hinge region/shortened CH2 domain has the ability to form one, two, or several covalent bonds.

In some embodiments, the covalent bond is a disulphide bridge.

In some embodiments, the immunoglobulin domain of the dimerization motif is a carboxyterminal C domain, such as a constant CH3 or CH4 domain, or a sequence that is substantially identical to said C domain or a variant or a functional fragment thereof.

In some embodiments, the carboxyterminal C domain is derived from IgY or IgG.

In some embodiments, the immunoglobulin domain of the dimerization motif has the ability to homodimerize.

In some embodiments, the immunoglobulin domain has the ability to homodimerize via noncovalent interactions. In some embodiments, the noncovalent interactions are hydrophobic interactions.

In some embodiments, the dimerization domain comprise two domains selected from CH2, and CH4 or functional fragment thereof, such as a CH2 and/or a CH4 domain or functional fragment thereof from IgY.

In some embodiments, the dimerization motif consist of hinge exons h1 and h4 connected through a linker to a C_{H}3 domain of human or mouse, IgG, such as IgG3 or shortened version of CH2 connected to C_{H}4 domain of avian IgY.

In some embodiments, the dimerization motif consist of an amino acid sequence having at least 80% sequence identity to the amino acid sequence of SEQ ID NO:13.

In some embodiments, the linker is a G₃S₂G₃SG linker.

In some embodiments, the antigenic unit and the dimerization motif is connected through a linker, such as a GLGGL linker or a GLSGL linker.

In some embodiments, the targeting unit consists of SEQ ID NO:11, or a variant thereof.

In some embodiments, the homodimeric protein binds specifically to chicken MHC class II molecule on avian cells with a K_{d} lower than 0.1 nM, such as lower than 50 pM, such as lower than 40, 30, 20 or 10 pM.

In some embodiments, the antigenic unit comprises an antigenic unit suitable for preventive and/or therapeutic effect for poultry diseases, such as an antigen selected from agents causing avian coccidiosis, avian encephalomyelitis, avian infectious bronchitis, such as S protein, S1 protein or S2 protein, avian infectious bursal disease, such as VP2 protein, avian reovirus, such as Sigma Cc protein, chicken anaemia virus, duck virus enteritis, egg drop syndrome 1976, erysipelas, infectious laryngotracheitis, Marek's disease, Newcastle disease, such as Hemagglutinin-neuraminidase fusion protein hemagglutinin-neuraminidase fusion protein, pasteurellosis, post-natal colibacillosis, salmonellosis, swollen head syndrome, turkey haemorrhagic enteritis, turkey rhinotracheitis and avian influenza, such as HA proteins, such as HA1, HA2, HA3, HA4, HA5, HA6, HA7, HA8, HA9, HA10, HA11, HA12, HA13, HA14, or HA15.

In some embodiments, the antigenic unit comprises an amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to any one amino acid sequence selected from SEQ ID NO:23 SEQ ID NO:30, SEQ ID NO:34, SEQ ID NO:36, SEQ ID NO:38, or SEQ ID NO:40, or a fragment thereof.

In some embodiments, the antigenic unit consists of an amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to any one amino acid sequence selected from SEQ ID NO:23 SEQ ID NO:30, SEQ ID NO:34, SEQ ID NO:36, SEQ ID NO:38, or SEQ ID NO:40, or a fragment thereof.

As used herein a fragment refers to a functional subsequence of a given sequence, usually with more than 10 amino acids, such as with more than 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 280, 300, 320, 340, 360, 380, 400, 420, 440, 460, 480, 500, 520, or 540 amino acids. For fragment used as antigenic units this fragment needs to be antigenic.

In some embodiments, the homodimeric protein according to the present invention is in its mature form without any signal peptide sequence.

In some embodiments, the avian are poultry, such as any one selected from Chicken, Duck, Emu, Goose, Indian Peafowl, Mute Swan, Ostrich, Pigeon, Turkey, Guineafowl, Common Pheasant, Golden Pheasant, and Rhea. In some embodiments, the avian is Chicken.

In some embodiments, the homodimeric protein according to the present invention comprises an amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to any one amino acid sequence selected from the hCH3 IgG3 domain of SEQ ID NO:17, the hinge region of SEQ ID NO:14, the hinge region of SEQ ID NO:15, the linker of SEQ ID NO:16, SEQ ID NO:18, or SEQ ID NO:19, the hinge region of SEQ ID NO:20 or SEQ ID NO:21, the CH3 hIgG3 domain of SEQ ID NO:22, the construct of SEQ ID NO:24, SEQ ID NO:31, or SEQ ID NO:32, the Chicken IgY CH2 domain of SEQ ID NO:25, SEQ ID NO:27, SEQ ID NO:28, or SEQ ID NO:29, the Chicken IgY CH4 of SEQ ID NO:26, or any functional fragment thereof.

Another aspect of the present invention relates to an amino acid chain comprising (1) an optional signal peptide, (2) a targeting unit, (3) a dimerization motif, and (4) an antigenic unit, said targeting unit being a scFv fragment specifically binding chicken MHC class II molecule on avian cells, which amino acid chain is able to form a homodimeric protein according to the invention. Another aspect of the present invention relates to a nucleic acid molecule, such as a DNA, encoding such amino acid chain. In some embodiments, the nucleic acid molecule is avian codon optimized.

Another aspect of the present invention relates to a nucleic acid molecule comprising or consisting of any one of nucleotide sequences selected from the list consisting of SEQ ID NO:6, SEQ ID NO:8, SEQ ID NO:10, nucleotides 1-36 of SEQ ID NO:12, nucleotides 37-81 of SEQ ID NO:12, nucleotides 82-111 of SEQ ID NO:12, nucleotides 112-432 of SEQ ID NO:12, nucleotides 433-447 of SEQ ID NO:12, or a variant thereof.

In some embodiments, the nucleic acid molecule according to the invention is comprised by a vector. In some embodiments, the nucleic acid molecule according to the invention is formulated for administration to a subject, such as an avian population to induce production of the homodimeric protein in said subject, such as an avian population.

Another aspect of the present invention relates to a homodimeric protein according to the invention, or an amino acid chain according to the invention, or the nucleic acid molecule according to the invention for use as a medicament.

Another aspect of the present invention relates to a pharmaceutical composition comprising the homodimeric protein according to the invention, or an amino acid chain according to the invention, or the nucleic acid molecule according to the invention.

Another aspect of the present invention relates to a host cell comprising the nucleic acid molecule according to the invention.

Another aspect of the present invention relates to a method for preparing a homodimeric protein according to the invention, or an amino acid chain according to the invention, the method comprising
transfecting the nucleic acid molecule according to the invention into a cell population;
culturing the cell population;
collecting and purifying the homodimeric protein, or amino acid chain expressed from the cell population.

A method for preparing a vaccine, such as a DNA vaccine, comprising an immunologically effective amount of a nucleic acid molecule according to the invention, the method comprising
preparing a nucleic acid molecule according to the invention;
dissolving the nucleic acid molecule obtained under step a) in a pharmaceutically acceptable carrier, diluent, or buffer.

Another aspect of the present invention relates to a vaccine against a disease or condition in an animal, such as a cancer or an infectious disease caused by a virus, bacteria or other infectious agent, the vaccine comprising an immunologically effective amount of a homodimeric protein according to the invention, or an amino acid chain according to the invention, or nucleic acid molecule, such as a DNA, according to the invention, wherein said vaccine is able to trigger both a T-cell- and B-cell immune response.

In some embodiments, the vaccine according to the invention further comprising a pharmaceutically acceptable carrier and/or adjuvant.

Another aspect of the present invention relates to a method of treating or preventing a disease or condition in an animal, such as a cancer or an infectious disease caused by a virus, bacteria or other infectious agent, the method comprising administering to said animal in need thereof, a homodimeric protein according to the invention, or an amino acid chain according to the invention, or the nucleic acid molecule, such as a DNA, according to the invention. In some embodiments, the method comprises administering to the subject, such as an avian population in need thereof of a nucleic acid molecule, such as a DNA, according to the invention with a subsequent step of electroporation. In some embodiments, the administration is performed intra-dermal or intra-muscular, respiratorial, mucosal, via the GI tract, or in ovo.

### Sequences:

### Oligonucleotide Sequences:

```
(SEQ ID NO: 1) IgκcDNAprimer:
(atcaggacagcaaagacagca) TGCTGTCTTTGCTGTCCTGAT
(SEQ ID NO: 4) 3′IgκC rev:
(CAAGAAGCACACGACTGAGGC ) gcctcagtcgtgtgcttcttg
(SEQ ID NO: 2) PolyG_NotI_frwd:
ATATGCGGCCGCGGGGGGGGGGGGGGGG
(SEQ ID NO: 3) 3′-mIgG1rev:
ttg acc agg cat ccc agg gtc
(SEQ ID NO: 5) VHL1:
ggt gtg cat tcc atg gac tgg acc tgg agg
SEQ ID NO: 6: 2G11 VH nucleotide sequence:
gaggtgaagctggtggagtcaggacctagcctcgtgaaaccttctcagactctgtccctcacctgttctgtcacgggcg
actccatcaccagtggttattggaactggatccggaaattcccagggaaaaaacttgaatacatggggttcataagcta
cagtggtgacacttattacaatccatctctcaaaagtcgaatctccatcactcgagacacatccaagaaccagtaccac
ctgcagttgaattctgtgacttctgaggacacagcaacatattactgtgcaagaaggaactacgttagtaactacgggg
ggtggtacttcggtgtctggggcgcagggaccacggtcaccgtctcctca
SEQ ID NO: 7: 2G11 VH amino acid sequence:
EVKLVESGPSLVKPSQTLSLTCSVTGDSITSGYWNWIRKFPGKKLEYMGFISYSGDTYYNPSLKSRISITRDTSKNQYH
LQLNSVTSEDTATYYCARRNYVSNYGGWYFGVWGAGTTVTVSS
SEQ ID NO: 8: 2G11 VL nucleotide sequence:
aacattgtaatgacccaatctcccaaatccatgtccatgtcagtaggagagagagtcaccctgagctgcaaggccagtg
agaatgtggttacttatgtatcctggtatcaacagaaaccagatcagtctcctaaactgctgatatacggggcatccaa
ccggtacactggggtccctgatcgcttcacaggcagtggatctgcaacagatttcactcttatcatcagcagtgttcag
gctgaagaccttgcagattatcactgtggacagagttacacctatcctcccacgttcggtgctgggaccaagctggagc
tgaaa
SEQ ID NO: 9: 2G11 VL amino acid sequence:
NIVMTQSPKSMSMSVGERVTLSCKASENVVTYVSWYQQKPDQSPKLLIYGASNRYTGVPDRFTGSGSATDFTLIISSVQ
AEDLADYHCGQSYTYPPTFGAGTKLELK
SEQ ID NO: 10: 2G11 ScFv nucleotide sequence:
aacattgtaatgacccaatctcccaaatccatgtccatgtcagtaggagagagagtcaccctgagctgcaaggccagtg
agaatgtggttacttatgtatcctggtatcaacagaaaccagatcagtctcctaaactgctgatatacggggcatccaa
ccggtacactggggtccctgatcgcttcacaggcagtggatctgcaacagatttcactcttatcatcagcagtgttcag
gctgaagaccttgcagattatcactgtggacagagttacacctatcctcccacgttcggtgctgggaccaagctggagc
tgaaaggtggaggcggatctggcggaggtggctctggcggtggcggatcggaggtgaagctggtggagtcaggacctag
cctcgtgaaaccttctcagactctgtccctcacctgttctgtcacgggcgactccatcaccagtggttattggaactgg
atccggaaattcccagggaaaaaacttgaatacatggggttcataagctacagtggtgacacttattacaatccatctc
tcaaaagtcgaatctccatcactcgagacacatccaagaaccagtaccacctgcagttgaattctgtgacttctgagga
cacagcaacatattactgtgcaagaaggaactacgttagtaactacggggggtggtacttcggtgtctggggcgcaggg
accacggtcaccgtctcctca
SEQ ID NO: 11: the 2G11 ScFv coding amino acid sequence (SEQ ID NO: 9 followed
by linker in bold and then SEQ ID NO: 7):
NIVMTQSPKSMSMSVGERVTLSCKASENVVTYVSWYQQKPDQSPKLLIYGASNRYTGVPDRFTGSGSATDFTLIISSVQ
AEDLADYHCGQSYTYPPTFGAGTKLELKGGGGSGGGGSGGGGSEVKLVESGPSLVKPSQTLSLTCSVTGDSITSGYWNW
IRKFPGKKLEYMGFISYSGDTYYNPSLKSRISITRDTSKNQYHLQLNSVTSEDTATYYCARRNYVSNYGGWYFGVWGAG
TTVTVSS
SEQ ID NO: 12 (Hinge h1 + hinge h4, Gly-Ser linker and Gly-Leu linker in bold
and hCH3 IgG3 domains suitable for constructs according to the present
invention)
GAGCTCAAAACCCCACTTGGTGACACAACTCACACAGAGCCCAAATCTTGTGACACACCTCCCCCGTGCCCAAGGTGCC
CAGGCGGTGGAAGCAGCGGAGGTGGAAGTGGAGGACAGCCCCGAGAACCACAGGTGTACACCCTGCCCCCATCCCGGGA
GGAGATGACCAAGAACCAGGTCAGCCTGACCTGCCTGGTCAAAGGCTTCTACCCCAGCGACATCGCCGTGGAGTGGGAG
AGCAGCGGGCAGCCGGAGAACAACTACAACACCACGCCTCCCATGCTGGACTCCGACGGCTCCTTCTTCCTCTACAGCA
AGCTCACCGTGGACAAGAGCAGGTGGCAGCAGGGGAACATCTTCTCATGCTCCGTGATGCATGAGGCTCTGCACAACCG
CTTCACGCAGAAGAGCCTCTCCCTGTCTCCGGGTAAA [symbol]
SEQ ID NO: 13 (Hinge h1 (SEQ ID NO: 14), Hinge h4 (SEQ ID NO: 15), linker in
bold and hCH3 IgG3 domains (SEQ ID NO: 17) suitable for constructs according
ato the present invention)
ELKTPLGDTTHTEPKSCDTPPPCPRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQKSLSLSPGKGLGGL
SEQ ID NO: 14: Hinge regions (IgG3 upper hinge), 12 amino acids:
ELKTPLGDTTHT
SEQ ID NO: 15: Hinge region (IgG3, lower hinge, 15 amino acids):
EPKSCDTPPPCPRCP
SEQ ID NO: 16: Gly-Ser Linker:
GGGSSGGGSG
SEQ ID NO: 17: hCH3 IgG3:
GQPREPQVYTLPPSREEMTKNQVSLTCLVKGFYPSDIAVEWESSGQPENNYNTTPPMLDSDGSFFLYSKLTVDKSRWQQ
GNIFSCSVMHEALHNRFTQKSLSLSPGK
SEQ ID NO: 18: Linker:
GLGGL
SEQ ID NO: 19: Linker:
GGGGSGGGGSGGGGS
SEQ ID NO: 20: Upper hinge hIgG3 (h1):
ELKTPLGDTTHT
SEQ ID NO: 21: Lower hinge hIgG3 (h4):
EPKSCDTPPPCPRCP
SEQ ID NO: 22: CH3 hIgG3:
GQPREPQVYTLPPSREEMTKNQVSLTCLVKGFYPSDIAVEWESSGQPENNYNTTPPMLDSDGSFFLYSKLTVDKSRWQQ
GNIFSCSVMHEALHNRFTQKSLSLSPGK
SEQ ID NO: 23: glycoprotein D from Herpes Simplex virus 2 with an optional
His-tag in bold:
MGRLTSGVGTAALLVVAVGLRVVCAKYALADPSLKMADPNRFRGKNLPVLDQLTDPPGVKRVYHIQPSLEDPFQPPSIP
ITVYYAVLERACRSVLLHAPSEAPQIVRGASDEARKHTYNLTIAWYRMGDNCAIPITVMEYTECPYNKSLGVCPIRTQP
RWSYYDSFSAVSEDNLGFLMHAPAFETAGTYLRLVKINDWTEITQFILEHRARASCKYALPLRIPPAACLTSKAYQQGV
TVDSIGMLPRFIPENQRTVALYSLKIAGWHGPKPPYTSTLLPPELSDTTNATQPELVPEDPEDSALLEDPAGTVSSQIP
PNWHIPSIQDVAPHHAPAAPSNPGLITGHHHHHH
SEQ ID NO: 24: Amino acid sequence of suitable chicken vaccibody construct
according to the invention: Targeting unit: anti-chicken MHC II scFv,
dimerization unit: human shortened hinge (h1 + h4), antigenic unit: gD from
herpes simplex virus 2 + an optional 6xHis-tag in bold. (In N- to C-terminal
order SEQ ID NO: 11 + SEQ ID NO: 14 + SEQ ID NO: 15 + SEQ ID NO: 16 +
SEQ ID NO: 17 + SEQ ID NO: 18 + SEQ ID NO: 23)
NIVMTQSPKSMSMSVGERVTLSCKASENVVTYVSWYQQKPDQSPKLLIYGASNRYTGVPDRFTGSGSATDFTLIISSVQ
AEDLADYHCGQSYTYPPTFGAGTKLELKGGGGSGGGGSGGGGSEVKLVESGPSLVKPSQTLSLTCSVTGDSITSGYWNW
IRKFPGKKLEYMGFISYSGDTYYNPSLKSRISITRDTSKNQYHLQLNSVTSEDTATYYCARRNYVSNYGGWYFGVWGAG
TTVTVSSELKTPLGDTTHTEPKSCDTPPPCPRCPGGGSSGGGSGGQPREPQVYTLPPSREEMTKNQVSLTCLVKGFYPS
DIAVEWESSGQPENNYNTTPPMLDSDGSFFLYSKLTVDKSRWQQGNIFSCSVMHEALHNRFTQKSLSLSPGKGLGGLMG
RLTSGVGTAALLVVAVGLRVVCAKYALADPSLKMADPNRFRGKNLPVLDQLTDPPGVKRVYHIQPSLEDPFQPPSIPIT
VYYAVLERACRSVLLHAPSEAPQIVRGASDEARKHTYNLTIAWYRMGDNCAIPITVMEYTECPYNKSLGVCPIRTQPRW
SYYDSFSAVSEDNLGFLMHAPAFETAGTYLRLVKINDWTEITQFILEHRARASCKYALPLRIPPAACLTSKAYQQGVTV
DSIGMLPRFIPENQRTVALYSLKIAGWHGPKPPYTSTLLPPELSDTTNATQPELVPEDPEDSALLEDPAGTVSSQIPPN
WHIPSIQDVAPHHAPAAPSNPGLITGHHHHHH
Amino acid sequences of avian IgY CH2 and CH4 domains
SEQ ID NO: 25: Chicken IgY CH2:
HPSSCTPSQSESVELLCLVTGFSPASAEVEWLVDGVGGLLVASQSPAVRSGSTYSLSSRVNVSGTDWREGKSYSCRVRH
PATNTVVEDHVKGCP
SEQ ID NO: 26: Chicken IgY CH4:
GPTTPPLIYPFAPHPEELSLSRVTLSCLVRGFRPRDIEIRWLRDHRAVPATEFVTTAVLPEERTANGAGGDGDTFFVYS
KMSVETAKWNGGTVFACMAVHEALPMRFSQRTLQKQAGK
SEQ ID NO: 27: Upper CH2 sequence IgY:
EWLVDGVGGL
SEQ ID NO: 28: Lower CH2 sequence IgY:
EGKSYSCRVRHPATNTVVEDHVKGCP
SEQ ID NO: 29: Extended lower CH2 sequence IgY:
VSGTDWREGKSYSCRVRHPATNTVVEDHVKGCP
SEQ ID NO: 30: Shortened HA5 with polybasic deletion from influenza
A/VietNam 1203/04 H5N1:
DQICIGYHANNSTEQVDTIMEKNVTVTHAQDILEKKHNGKLCDLDGVKPLILRDCSVAGWLLGNPMCDEFINVPEWSYI
VEKANPVNDLCYPGDFNDYEELKHLLSRINHFEKIQIIPKSSWSSHEASLGVSSACPYQGKSSFFRNVVWLIKKNSTYP
TIKRSYNNTNQEDLLVLWGIHHPNDAAEQTKLYQNPTTYISVGTSTLNQRLVPRIATRSKVNGQSGRMEFFWTILKPND
AINFESNGNFIAPEYAYKIVKKGDSTIMKSELEYGNCNTKCQTPMGAINSSMPFHNIHPLTIGECPKYVKSNRLVLATG
LRNSPQRERRRKKRGLFGAIAGFIEGGWQGMVDGWYGYHHSNEQGSGYAADKESTQKAIDGVTNKVNSIIDKMNTQFEA
VGREFNNLERRIENLNKKMEDGFLDVWTYNAELLVLMENERTLDFHDSNVKNLYDKVRLQLRDNAKELGNGCFEFYHKC
DNECMESVRNGTYDYPQYSEEARLKREEISGVKLESIGIYQILSIYSTVASSL
SEQ ID NO: 31: Amino acid sequence of suitable chicken vaccibody construct
according to the invention:: Targeting unit: anti-chicken MHC II, dimerization
unit: shortened CH2 (lower + upper CH2) + entire CH4, antigenic unit: H5 from
influenza. (In N- to C-terminal order SEQ ID NO: 11 + SEQ ID NO: 27 + SEQ ID
NO: 28 + SEQ ID NO: 16 + SEQ ID NO: 26 + SEQ ID NO: 18 + SEQ ID NO: 30)
NIVMTQSPKSMSMSVGERVTLSCKASENVVTYVSWYQQKPDQSPKLLIYGASNRYTGVPDRFTGSGSATDFTLIISSVQ
AEDLADYHCGQSYTYPPTFGAGTKLELKGGGGSGGGGSGGGGSEVKLVESGPSLVKPSQTLSLTCSVTGDSITSGYWNW
IRKFPGKKLEYMGFISYSGDTYYNPSLKSRISITRDTSKNQYHLQLNSVTSEDTATYYCARRNYVSNYGGWYFGVWGAG
TTVTVSSEWLVDGVGGLEGKSYSCRVRHPATNTVVEDHVKGCPGGGSSGGGSGGPTTPPLIYPFAPHPEELSLSRVTLS
CLVRGFRPRDIEIRWLRDHRAVPATEFVTTAVLPEERTANGAGGDGDTFFVYSKMSVETAKWNGGTVFACMAVHEALPM
RFSQRTLQKQAGKGLGGLDQICIGYHANNSTEQVDTIMEKNVTVTHAQDILEKKHNGKLCDLDGVKPLILRDCSVAGWL
LGNPMCDEFINVPEWSYIVEKANPVNDLCYPGDFNDYEELKHLLSRINHFEKIQIIPKSSWSSHEASLGVSSACPYQGK
SSFFRNVVWLIKKNSTYPTIKRSYNNTNQEDLLVLWGIHHPNDAAEQTKLYQNPTTYISVGTSTLNQRLVPRIATRSKV
NGQSGRMEFFWTILKPNDAINFESNGNFIAPEYAYKIVKKGDSTIMKSELEYGNCNTKCQTPMGAINSSMPFHNIHPLT
IGECPKYVKSNRLVLATGLRNSPQRERRRKKRGLFGAIAGFIEGGWQGMVDGWYGYHHSNEQGSGYAADKESTQKAIDG
VTNKVNSIIDKMNTQFEAVGREFNNLERRIENLNKKMEDGFLDVWTYNAELLVLMENERTLDFHDSNVKNLYDKVRLQL
RDNAKELGNGCFEFYHKCDNECMESVRNGTYDYPQYSEEARLKREEISGVKLESIGIYQILSIYSTVASSL
SEQ ID NO: 32: Variant 2 with an extended lower CH2 sequence:
(In N- to C-terminal order SEQ ID NO: 11 + SEQ ID NO: 29 + SEQ ID NO:
16 + SEQ ID NO: 26 + SEQ ID NO: 18 + SEQ ID NO: 30)
NIVMTQSPKSMSMSVGERVTLSCKASENVVTYVSWYQQKPDQSPKLLIYGASNRYTGVPDRFTGSGSATDFTLIISSVQ
AEDLADYHCGQSYTYPPTFGAGTKLELKGGGGSGGGGSGGGGSEVKLVESGPSLVKPSQTLSLTCSVTGDSITSGYWNW
IRKFPGKKLEYMGFISYSGDTYYNPSLKSRISITRDTSKNQYHLQLNSVTSEDTATYYCARRNYVSNYGGWYFGVWGAG
TTVTVSSVSGTDWREGKSYSCRVRHPATNTVVEDHVKGCPGGGSSGGGSGGPTTPPLIYPFAPHPEELSLSRVTLSCLV
RGFRPRDIEIRWLRDHRAVPATEFVTTAVLPEERTANGAGGDGDTFFVYSKMSVETAKWNGGTVFACMAVHEALPMRFS
QRTLQKQAGKGLGGLDQICIGYHANNSTEQVDTIMEKNVTVTHAQDILEKKHNGKLCDLDGVKPLILRDCSVAGWLLGN
PMCDEFINVPEWSYIVEKANPVNDLCYPGDFNDYEELKHLLSRINHFEKIQIIPKSSWSSHEASLGVSSACPYQGKSSF
FRNVVWLIKKNSTYPTIKRSYNNTNQEDLLVLWGIHHPNDAAEQTKLYQNPTTYISVGTSTLNQRLVPRIATRSKVNGQ
SGRMEFFWTILKPNDAINFESNGNFIAPEYAYKIVKKGDSTIMKSELEYGNCNTKCQTPMGAINSSMPFHNIHPLTIGE
CPKYVKSNRLVLATGLRNSPQRERRRKKRGLFGAIAGFIEGGWQGMVDGWYGYHHSNEQGSGYAADKESTQKAIDGVTN
KVNSIIDKMNTQFEAVGREFNNLERRIENLNKKMEDGFLDVWTYNAELLVLMENERTLDFHDSNVKNLYDKVRLQLRDN
AKELGNGCFEFYHKCDNECMESVRNGTYDYPQYSEEARLKREEISGVKLESIGIYQILSIYSTVASSL
SEQ ID NO: 33: Infectious bursal disease virus viral protein 2, viral protein 4
and viral protein 3 of segment A (VP2-VP4-VP3); References: 1) De Schutter K.,
Lin Y. -C., Tiels P, et al. (2009). “Genome sequence of the recombinant protein
production host Pichia pastoris.”. Nature Biotechnology 27 (6): 561-566, 2) Daly
R, Hearn MT (2005). “Expression of heterologous proteins in Pichia pastoris: a
useful experimental tool in protein engineering and production”. 3. Mol.
Recognit. 18 (2): 119-38, 3) Pitcovski 3., Gutter B., Gallili G., et al. (2003).
“Development and large-scale use of recombinant VP2 vaccine for the prevention
of infectious bursal disease of chickens”. Vaccine 21 4736-43, DNA Sequence:
ATGACAAACCTGCAAGATCAAACCCAACAGATTGTTCCGTTCATACGGAGCCTTCTGATGCCAACAACCGGACCGGCGT
CCATTCCGGACGACACCCTAGAGAAGCACACTCTCAGGTCAGAGACCTCGACCTACAATTTGACTGTGGGGGACACAGG
GTCAGGGCTAATTGTCTTTTTCCCTGGTTTCCCTGGCTCAATTGTGGGTGCTCACTACACACTGCAGAGCAATGGGAAC
TACAAGTTCGATCAGATGCTCCTGACTGCCCAGAACCTACCGGCCAGCTACAACTACTGCAGGCTAGTGAGTCGGAGTC
TCACAGTGAGGTCAAGCACACTCCCTGGTGGCGTTTATGCACTAAATGGCACCATAAACGCCGTGACCTTCCAAGGAAG
CCTGAGTGAACTGACAGATGTTAGCTACAATGGGTTGATGTCTGCAACAGCCAACATCAACGACAAAATCGGGAACGTC
CTAGTAGGGGAAGGGGTAACCGTCCTCAGCTTACCCACATCATATGATCTTGGGTATGTGAGACTCGGTGACCCCATTC
CCGCTATAGGGCTCGACCCAAAAATGGTAGCAACATGTGACAGCAGTGACAGGCCCAGAGTCTACACCATAACTGCAGC
CGATGATTACCAATTCTCATCACAGTACCAGGCAGGTGGGGTAACAATCACACTGTTCTCAGCTAATATCGATGCCATC
ACAAGCCTCAGCATTGGGGGAGAACTCGTGTTCCAAACAAGCGTCCAAGGCCTTATACTGGGTGCTACCATCTACCTTA
TAGGCTTTGATGGGACTGCGGTAATCACCAGAGCTGTGGCCGCAGACAATGGGCTAACGGCCGGCACTGACAACCTTAT
GCCATTCAATATTGTGATTCCAACCAGCGAGATAACCCAGCCAATCACATCCATCAAACTGGAGATAGTGACCTCCAAA
AGTGGTGGTCAGGCGGGGGACCAGATGTCATGGTCAGCAAGTGGGAGCCTAGCAGTGACGATCCACGGTGGCAACTATC
CAGGGGCCCTCCGTCCCGTCACACTAGTAGCCTACGAAAGAGTGGCAACAGGATCTGTCGTTACGGTCGCCGGGGTGAG
CAACTTCGAGCTGATCCCAAATCCTGAACTAGCAAAGAACCTGGTCACAGAATACGGCCGATTTGACCCAGGAGCCATG
AACTACACAAAATTGATACTGAGTGAGAGGGACCGTCTTGGCATCAAGACCGTATGGCCAACAAGGGAGTACACTGACT
TTCGCGAGTACTTCATGGAGGTGGCCGACCTCAACTCTCCCCTGAAGATTGCAGGAGCATTTGGCTTCAAAGACATAAT
CCGGGCCATAAGGAGGATAGCTGTGCCGGTGGTCTCTACATTGTTCCCACCCGCCGCTCCCCTAGCCCATGCAATTGGG
GAAGGTGTAGACTACCTGCTGGGCGATGAGGCACAGGCTGCTTCAGGAACTGCTCGAGCCGCGTCAGGAAAAGCAAGAG
CTGCCTCAGGCCGCATAAGGCAGCTAACTCTCGCCGCCGACAAGGGGTACGAGGTAGTCGCGAATCTGTTTCAGGTGCC
CCAGAATCCTGTAGTCGACGGGATTCTCGCTTCACCTGGGATACTCCGCGGTGCACACAACCTCGACTGCGTGTTGAGA
GAGGGTGCCACGCTATTCCCTGTGGTCATCACGACAGTGGAAGATGCCATGACACCCAAAGCATTGAACAGCAAAATGT
TTGCTGTCATTGAAGGCGTGCGAGAAGATCTACAACCTCCATCTCAAAGAGGATCCTTCATACGAACTCTCTCCGCACA
TAGAGTCTATGGATATGCTCCAGATGGGGTACTTCCACTGGAGACTGGGAGAGATTACACCGTGGTCCCAATAGATGAT
GTCTGGGATGACAGCATTATGCTGTCCAAAGACCCCATACCTCCTATTGTGGGAAACAGTAGAAACCTAGACATAGCTT
ACATGGATGTGTTTCGACCCAAAGTCCCCATCCATGTGGCCATGACGGGAGCCCTCAATGCCTATGGCGAGATTGAGAA
CGTGAGCTTTAGAAGCACCAAGCTCGCCACTGCACACCGACTTGGCCTCAAGTTGGCTGGTCCCGGTGCATTTGACGTG
AACACCGGGTCCAACTGGGCAACGTTCATCAAACGTTTCCCTCACAATCCACGAGACTGGGACAGGCTCCCTTACCTCA
ACCTTCCATACCTTCCACCCAATGCAGGACGCCAGTACGACCTGGCCATGGCTGCTTCAGAGTTCAAAGAGACCCCCGA
ACTCGAGAGCGCCGTCAGAGCCATGGAAGCAGAAGCCAACGTGGACCCACTGTTCCAATCTGCACTCAGCGTGTTCATG
TGGCTGGAAGAGAATGGGATTGTGACTGATATGGCCAACTTCGCACTCAGCGACCCGAATGCCCATCGGATGCGCAATT
TTCTCGCAAACGCACCACAAGCAGGCAGCAAGTCGCAAAGAGCCAAGTACGGGACAGCAGGCTACGGAGTGGAGGCCCG
GGGCCCCACTCCAGAGGAAGCACAGAGGGAAAAAGACACACGGATCTCAAAGAAGATGGAGACTATGGGCATCTACTTT
GCAACACCAGAATGGGTAGCACTCAATGGGCACCGGGGGTCAAGCCCCGGCCAGCTAAAGTACTGGCAGAACACACGAG
AAATACCTGATCCAAACGAGGACTACCTAGACTACGTGCATGCAGAGAAGAGCCGGTTGGCATCAGAAGAACAAATCCT
AAGGGCAGCCACGTCGATCTACGGGGCTCCAGGACAGGCTGAGCCACCCCAGGCCTTCATAGACGAAGTCGCCAAAGTC
TATGAAATCAACCATGGGCGTGGCCCCAACCAAGAACAGATGAAAGATCTGCTCTTGACTGCGATGGAGATGAAGCATC
GCAATCCCAGGCGGGCTCCACCAAAGCCCAAGCCAAAACCCAATGTTCCAACACAGAGACCCCCTGGTCGGCTGGGCCG
CTGGATCAGGGCTGTCTCTGATGAGGACCTTGAGTGA
SEQ ID NO: 34: Amino Acid sequence:
MTNLQDQTQQIVPFIRSLLMPTTGPASIPDDTLEKHTLRSETSTYNLTVGDTGSGLIVFFPGFPGSIVGAHYTLQSNGN
YKFDQMLLTAQNLPASYNYCRLVSRSLTVRSSTLPGGVYALNGTINAVTFQGSLSELTDVSYNGLMSATANINDKIGNV
LVGEGVTVLSLPTSYDLGYVRLGDPIPAIGLDPKMVATCDSSDRPRVYTITAADDYQFSSQYQAGGVTITLFSANIDAI
TSLSIGGELVFQTSVQGLILGATIYLIGFDGTAVITRAVAANNGLTAGTDNLMPFNIVIPTSEITQPITSIKLEIVTSK
SGGQAGDQMSWSASGSLAVTIHGGNYPGALRPVTLVAYERVATGSVVTVAGVSNFELIPNPELAKNLVTEYGRFDPGAM
NYTKLILSERDRLGIKTVWPTREYTDFREYFMEVADLNSPLKIAGAFGFKDIIRALRRIAVPVVSTLFPPAAPL
SEQ ID NO: 35: Influenza A virus (A/chicken/Israel/215/2007(H9N2)) segment 4
hemagglutinin (HA); Reference: GenBank: FJ464716.1, DNA sequence:
      1 atggaaataa tatcactgat gactatacta ctagtagtaa caacaagcaa tgcagataaa
    61 atatgcattg gccaccagtc aacaaattcc acagaaactg tggatacact aacagaaact
  121 aatgttcctg tgacacatgc caaagaattg ctccacacag agcacaatgg aatgctgtgt
  181 gcaacaaatc tgggaaatcc cctcatccta gacacatgca ctatcgaagg acttatctat
  241 ggtaaccctt cttgtgacat gttgttgggg ggaagggaat ggtcctacat cgttgaaaga
  301 ccatcagcgg taaatggaac atgttaccct gggaatgtgg aaaacttaga ggaactcaga
  361 acacttttta gttcctctag ttcctatcaa agaatccaaa tattcccaga cacaatctgg
  421 aatgtgactt acaatggaac aagcaaatca tgttcaaatt cattctacag gaatatgaga
  481 tggctaactc aaaagaacgg ggtttatcct gttcaagacg cccaatacac aaataatcgg
  541 ggaaaggaca ttcttttcgt gtggggcata catcatccac ccaccgatac tgcacagacg
  601 aatttgtaca caagaaccga cacaacaaca agcgtaacaa cagaaaattt agataggacc
  661 ttcaaaccat tgatagggcc aaggcccctt gtcaatggtc tgattggaag aattaattat
  721 tattggtcgg tactaaaacc aggccagaca ttgcgagtaa gatccaatgg gaatctaatt
  781 gctccatggt ttggacacgt tctctcagga gagagccatg ggagaatcct gaaaactgat
  841 ttaaacagtg gtaattgtgt agtgcaatgt cagactgaaa aaggtggcct aaacagtaca
  901 ttgcctttcc acaatatcag taaatatgca tttgggaatt gccccaaata tattggagtc
  961 aagagtctca aactggcaat cggtctgaga aacgtgcctg ccaggtcaag tagaggacta
1021 tttggagcca tagctggatt catagaagga ggttggccag ggctagtcgc cggttggtat
1081 ggtttccagc attcaaatga tcaaggggtt ggtatggctg cagataggga ttcaactcaa
1141 aaggcagttg acaaaataac atccaaggtg aataatatag tcgacaagat gaacaagcaa
1201 tatgaaataa ttgatcatga attcagtgag gttgaaacta gactcaatat gatcaataac
1261 aagattgatg accaaataca agatgtatgg gcatataatg cagagttgct agtactactt
1321 gagaaccaga aaacactcga tgagcatgac gcaaacgtga acaacctata taacaaggtg
1381 aaaagggcct tgggctccaa tgcgatggaa gatgggaagg gctgtttcga gctataccac
1441 aaatgtgatg accaatgcat ggaaactatt cggaacggga cctataatag gagaaagtac
1501 aaagaggaat caagactaga aaggcagaaa atagagggag tcaaactgga atctgagggg
1561 atttacaaaa tacttaccat ttattcgact gtcgcctcat ctcttgtact tgcaatgggg
1621 tttgctgcct tcttattctg ggccatgtcc aatggatcat gcaggtgcaa catctgtata
1681 taattagcaa aaacaccctt gtttcta
SEQ ID NO: 36: Amino acid sequence:
MEIISLMTILLVVTTSNADKICIGHQSTNSTETVDTLTETNVPVTHAKELLHTEHNGMLCATNLGNPLILDTCTIEGLI
YGNPSCDMLLGGREWSYIVERPSAVNGTCYPGNVENLEELRTLFSSSSSYQRIQIFPDTIWNVTYNGTSKSCSNSFYRN
MRWLTQKNGVYPVQDAQYTNNRGKDILFVWGIHHPPTDTAQTNLYTRTDTTTSVTTENLDRTFKPLIGPRPLVNGLIGR
INYYWSVLKPGQTLRVRSNGNLIAPWFGHVLSGESHGRILKTDLNSGNCVVQCQTEKGGLNSTLPFHNISKYAFGNCPK
YIGVKSLKLAIGLRNVPARSSRGLFGAIAGFIEGGWPGLVAGWYGFQHSNDQGVGMAADRDSTQKAVDKITSKVNNIVD
KMNKQYEIIDHEFSEVETRLNMINNKIDDQIQDVWAYNAELLVLLENQKTLDEHDANVNNLYNKVKRALGSNAMEDGKG
CFELYHKCDDQCMETIRNGTYNRRKYKEESRLERQKIEGVKLESEGIYKILTIYSTVASSLVLAMGFAAFLFWAMSNGS
CRCNICI
SEQ ID NO: 37: Avian infectious bronchitis virus isolate variant 1 51 spike
glycoprotein gene, Reference: GenBank: AF093795.1; DNA sequence:
      1 atgttgggca aaccgctttt actagtgact ctttggtatg cactatgtag tgctttgctc
    61 tatgatagta gtacttacgt ttactactac caaagtgctt ttaggcctag ttcaggttgg
  121 cacatacatg ggggtgctta tgcagtagat agggttttta atgaaaccaa caatgcaggc
  181 agtgtatctg attgcactgc tggtactttt tatgaaagcc ataatatttc tgctgtttct
  241 gtagccatga cagcaccaca taatggtatg tcttggtcag tttcacaatt ttgtacagct
  301 cattgtaact tctcagactt tacagtgttc gttacgcatt gttttaaaaa tcaacctggt
  361 agttgtccct tgacaggtat gattcctcag aatcatattc gtatttctgc tatgagacaa
  421 ggaactttgt tttataactt aacagttagt gtgtctaaat atcctagatt taaatcgctt
  481 caatgtgtta gcaattctac atctgtctat gtaaatggtg atcttgtttt cacttctaat
  541 gaaacttctt acattacggg tgcaggcgtt tattttaaaa gtggtgggcc tgtaacttat
  601 aaagttatga aagaagttaa agccctagcc tactttatta atggtaccgc acaagaggtt
  661 attttatgtg ataactcacc tagaggtttg cttgcatgtc agtataatac tggtaatttt
  721 tcagatggat tctacccttt tactaatcat tctttagtta aggataggtt tattgtatat
  781 cgagaaagtg gcactaacac tactttaaag ttaactaatt tcagttttac taatgtaagt
  841 aatgctcctc ctaattcagg tggcgttgat actttccaat tatatcaaac acatactgct
  901 caggatggtt attataattt taatttatca tttctgagta gttttgtgta taaaccatct
  961 gattttatgt atgggtcata ccacccacat tgtaatttta gaccagaaaa tattaataat
1021 ggcttatggt ttaattcatt atctgtgtca cttacttacg gacccattca aggtggttgt
1081 aagcaatctg tttttaataa tagagcaact tgttgctatg cttattctta tcaagggcct
1141 agtttatgta agggtgttta tagaggggag ctaatgcaat actttgaatg tggacttcta
1201 gtttacgtaa ctaagagtga tggctctcgt atacaaacta gaagtgaacc acttgtgtta
1261 actcaatata attataacaa cattacttta aacaagtgtg ttgagtataa tatatatggt
1321 agagttggtc aaggtcttat tactaatgta actgaagcaa ctgctaatta tagttatcta
1381 gcagatggtg gtttagctat tttagatacc tcaggagcca tagacatatt tgttgttcaa
1441 ggtgcacatg gtcttaatta ttataaggtt aatccctgtg aggatgttaa ccaacagttt
1501 gtagtgtcag gtggcaactt agttggcatt cttacatctc ataatgaaac aggttctgaa
1561 tctattgaga accagtttta catcaaactc actaacggaa cacgtcgctc tagacgt
SEQ ID NO: 38: Amino acid sequence:
MLGKPLLLVTLWYALCSALLYDSSTYVYYYQSAFRPSSGWHIHGGAYAVDRVFNETNNAGSVSDCTAGTFYESHNISAV
SVAMTAPHNGMSWSVSQFCTAHCNFSDFTVFVTHCFKNQPGSCPLTGMIPQNHIRISAMRQGTLFYNLTVSVSKYPRFK
SLQCVSNSTSVYVNGDLVFTSNETSYITGAGVYFKSGGPVTYKVMKEVKALAYFINGTAQEVILCDNSPRGLLACQYNT
GNFSDGFYPFTNHSLVKDRFIVYRESGTNTTLKLTNFSFTNVSNAPPNSGGVDTFQLYQTHTAQDGYYNFNLSFLSSFV
YKPSDFMYGSYHPHCNFRPENINNGLWFNSLSVSLTYGPIQGGCKQSVFNNRATCCYAYSYQGPSLCKGVYRGELMQYF
ECGLLVYVTKSDGSRIQTRSEPLVLTQYNYNNITLNKCVEYNIYGRVGQGLITNVTEATANYSYLADGGLAILDTSGAI
DIFVVQGAHGLNYYKVNPCEDVNQQFVVSGGNLVGILTSHNETGSESIENQFYIKLTNGTRRSRR
SEQ ID NO: 39: Eimeria maxima 56 kDa gametocyte antigen (gam56) Reference:
GenBank: AY129951.2; DNA sequence:
      1 agcagaacat agggagttca tctgttcctt cttttcatca tttattcctc gtttctcacc
    61 gttttatttt ttttgtgtaa ccctctccgc tgttgagtcc caatgacccg cctcggcctc
  121 gctgctgtcg cgctggctct cgccgtgggc ccttccatgg cagtgcccag caccactcct
  181 gttgagaacc aggttcaccc ttacagcgag atgagtacct accaggaggg gagtgccccg
  241 ggggctccgg aggacaccac caccaccact acgtcgtccc ctgtttccga tggagccgag
  301 cagtggcttg agagctttgt tcgtgctgtg cagcgccagc tgcagcttca ggaccaaatg
  361 atgcgtcagc tcatgaggga cattcaggag tacctgagca ctgcgttcaa ctgggcagag
  421 aaccagtcta ctgcctacac ccgtgttacc gagatgatgg acatgatctc caacagaatg
  481 aacgctgcca tggacagctc aaacgaactc atgaccacta gcgacaccac agaccccgag
  541 accctccgcc gtgcaactcg caagtacatg aaggaggttc gcgttcagga cgtcctggta
  601 gatgctctct gggcctctct ccgcggtgta cagacagctg cctggatgaa tggagtgacc
  661 gctattgaga aggaggagac gactcccatg gctagccgcg ctgctgagga gttcctccac
  721 cgcatgtacc ataacctgag ggcagcaggt atgtctgaag aagatgttgc caagttcatc
  781 cctagagccg agtacaaccc ctccgagcag tcaagaaata tgggcagaaa gggcaggagc
  841 ttctactacg gcggctatcc cagctactac aactccccct actacagcta cagcagctac
  901 cccagctact acaactacag ctacccgtca tacagctaca gcagctaccc cagctactac
  961 cgctacagca gctaccccta ctacaactac agctatccca gctactacaa ctacggcagc
1021 tacccctact acagttatag cagctacccc agctggtact ggcgccgtct ccgctctttg
1081 gcaacagcaa cttgcccaga ctgccctcct ctcaccactc ccagcatgat cccaactccc
1141 cccccaatga tgaacatgat gaacacccca ccccccatgg caaacatgat gaccagcatg
1201 atgatgaaca ctcccatggt tcctcctccc cgcaccctcg gaactgaagc catgagcctc
1261 ggcttggccc ccatcggtat caccggcgcc cccatgacag gtttcggtgt tcctcctgag
1321 ttcggtccct ttggagccga aggtatcggc ctccccaccg atgccctcgg cagcaccccc
1381 gaaatgacac cattcgaccc aactaccccc tacagaactc tcgcccccat ggacctcccc
1441 cccatccccc ctcctgtctt ccctgaaacc cctatgaggc cacctactcc cttcggcttc
1501 ggacctgcac ctgttcctcc catgcccttc taaacgacct accatccctc aatccatagc
1561 tcacatttcg tagcctcaaa acagtttttt gttcatttca cttccaggac tcatgctgcg
1621 acatttgcat tcgtacctcg aaaccgtcaa cctcaaaccc caaaccattc tgtgacctcc
1681 cctcgcaaac gcggaaggcg gaacattttt tctgaagtat attactacgt taaaaaaaaa
1741 aaaaaaaaaa aaaa
SEQ ID NO: 40: Amino acid sequence:
MTRLGLAAVALALAVGPSMAVPSTTPVENQVHPYSEMSTYQEGSAPGAPEDTTTTTTSSPVSDGAEQWLESFVRAVQRQ
LQLQDQMMRQLMRDIQEYLSTAFNWAENQSTAYTRVTEMMDMISNRMNAAMDSSNELMTTSDTTDPETLRRATRKYMKE
VRVQDVLVDALWASLRGVQTAAWMNGVTAIEKEETTPMASRAAEEFLHRMYHNLRAAGMSEEDVAKFIPRAEYNPSEQS
RNMGRKGRSFYYGGYPSYYNSPYYSYSSYPSYYNYSYPSYSYSSYPSYYRYSSYPYYNYSYPSYYNYGSYPYYSYSSYP
SWYWRRLRSLATATCPDCPPLTTPSMIPTPPPMMNMMNTPPPMANMMTSMMMNTPMVPPPRTLGTEAMSLGLAPIGITG
APMTGFGVPPEFGPFGAEGIGLPTDALGSTPEMTPFDPTTPYRTLAPMDLPPIPPPVFPETPMRPPTPFGFGPAPVPPM
PF
```

### EXAMPLES

### Example 1

### Construction of 2G11-Vaccibody

For utilising the antibody 2G11 as a targeting unit in a vaccibody vaccine, the coding region of the antibody constituting the antigen-binding site must be isolated, verified and cloned into the vaccibody format. The antigen binding parts of antibodies are the variable domains composed of a heavy and a light chain. These domains are denoted VH and VL, respectively. In the vaccibody format two amino acid chains are folded into a dimer. Therefore, the desired form of a VH and VL antibody fragment is a ScFv fragment which is composed of the VL and VH combined through a flexible linker.

mRNA, cDNA Synthesis and RT PCR of 2G11 VH and VL Genes:

### Oligonucleotide Sequences:

IgκcDNAprimer:
(SEQ ID NO: 1)
(atcaggacagcaaagacagca) TGCTGTCTTTGCTGTCCTGAT
3′IgκC rev:
(SEQ ID NO: 4)
(CAAGAAGCACACGACTGAGGC) gcctcagtcgtgtgcttcttg
PolyG_NotI_frwd:
(SEQ ID NO: 2)
ATATGCGGCCGCGGGGGGGGGGGGGGGG
3′-mIgG1rev:
(SEQ ID NO: 3)
ttg acc agg cat ccc agg gtc
VHL1:
(SEQ ID NO: 5)
ggt gtg cat tcc atg gac tgg acc tgg agg

RNA was isolated from the MaD2G11 hybridoma (Salomonsen et. al. Immunogenetics 1987; 25(6):373-82) cells, by using the Absolutely RNA® Miniprep Kit (Stratagene). The cDNA synthesis was performed according to protocol using the IgKcDNAprimer (SEQ ID NO:1) and poly dCTP 3′-tailing of the cDNA using terminal transferase was performed by mixing 10 μl cDNA (unknown concentration), 2 μl 1×TdT reaction buffer, 2 μl CoCl₂, 1 μl dCTP, 2 μl rTdT and 4 μl ddH₂O and incubating the mixture for 15 min at 37° C. Then the solution was placed on ice, glycogen precipitated as in step 5 and rehydrated in 20 μl dH₂O on ice (see e.g. Nilssen N R et al. Nucleic Acids Res. 2012 September; 40(16):e120). PCR reaction for amplification of variable light (VL) genes was performed by using the oligonucleotides PolyG NotI frwd (SEQ ID NO:2) and oligonucleotides compatible to mouse Constant Kappa (SEQ ID NO:4), while the variable heavy (VH) genes was synthesized using oligonucleotides compatible with the VH leader (SEQ ID NO:5) and mouse IgG1 constant region (SEQ ID NO:3) sequences.

The PCR products were further cloned into vectors by TOPO-cloning according to the manufacturers description (Zero Blunt® TOPO PCR Cloning Kit, Invitrogen).

### Verification of 2G11 VH and VL Sequences:

Seven isolated plasmids form each of the VL and VH cloning procedures were sequenced and productive VL and VH sequences were verified by homology search (IMGT/V QUEST).

SEQ ID NO: 6: 2G11 VH nucleotide sequence:
gaggtgaagctggtggagtcaggacctagcctcgtgaaaccttctcaga
ctctgtccctcacctgttctgtcacgggcgactccatcaccagtggtta
ttggaactggatccggaaattcccagggaaaaaacttgaatacatgggg
ttcataagctacagtggtgacacttattacaatccatctctcaaaagtc
gaatctccatcactcgagacacatccaagaaccagtaccacctgcagtt
gaattctgtgacttctgaggacacagcaacatattactgtgcaagaagg
aactacgttagtaactacggggggtggtacttcggtgtctggggcgcag
ggaccacggtcaccgtctcctca
SEQ ID NO: 7: 2G11 VH amino acid sequence:
EVKLVESGPSLVKPSQTLSLTCSVTGDSITSGYWNWIRKFPGKKLEYMG
FISYSGDTYYNPSLKSRISITRDTSKNQYHLQLNSVTSEDTATYYCARR
NYVSNYGGWYFGVWGAGTTVTVSS
SEQ ID NO: 8: 2G11 VL nucleotide sequence:
aacattgtaatgacccaatctcccaaatccatgtccatgtcagtaggag
agagagtcaccctgagctgcaaggccagtgagaatgtggttacttatgt
atcctggtatcaacagaaaccagatcagtctcctaaactgctgatatac
ggggcatccaaccggtacactggggtccctgatcgcttcacaggcagtg
gatctgcaacagatttcactcttatcatcagcagtgttcaggctgaaga
ccttgcagattatcactgtggacagagttacacctatcctcccacgttc
ggtgctgggaccaagctggagctgaaa
SEQ ID NO: 9: 2G11 VL amino acid sequence:
NIVMTQSPKSMSMSVGERVTLSCKASENVVTYVSWYQQKPDQSPKLLIY
GASNRYTGVPDRFTGSGSATDFTLIISSVQAEDLADYHCGQSYTYPPTF
GAGTKLELK

### Design of 2G11 Single Chain Fv (ScFv) Construct:

The following ScFv was constructed.

### SEQ ID NO:33: 2G11 ScFv Nucleotide Sequence Including Restriction Enzyme Sites:

tgcattccaacattgtaatgacccaatctcccaaatccatgtccatgtc
agtaggagagagagtcaccctgagctgcaaggccagtgagaatgtggtt
acttatgtatcctggtatcaacagaaaccagatcagtctcctaaactgc
tgatatacggggcatccaaccggtacactggggtccctgatcgcttcac
aggcagtggatctgcaacagatttcactcttatcatcagcagtgttcag
gctgaagaccttgcagattatcactgtggacagagttacacctatcctc
ccacgttcggtgctgggaccaagctggagctgaaaggtggaggcggatc
tggcggaggtggctctggcggtggcggatcggaggtgaagctggtggag
tcaggacctagcctcgtgaaaccttctcagactctgtccctcacctgtt
ctgtcacgggcgactccatcaccagtggttattggaactggatccggaa
attcccagggaaaaaacttgaatacatggggttcataagctacagtggt
gacacttattacaatccatctctcaaaagtcgaatctccatcactcgag
acacatccaagaaccagtaccacctgcagttgaattctgtgacttctga
ggacacagcaacatattactgtgcaagaaggaactacgttagtaactac
ggggggtggtacttcggtgtctggggcgcagggaccacggtcaccgtct
cctcaggtgagtcgtacg
SEQ ID NO: 11: 2G11 ScFv coding amino acid
sequence:
NIVMTQSPKSMSMSVGERVTLSCKASENVVTYVSWYQQKPDQSPKLLIY
GASNRYTGVPDRFTGSGSATDFTLIISSVQAEDLADYHCGQSYTYPPTF
GAGTKLELKGGGGSGGGGSGGGGSEVKLVESGPSLVKPSQTLSLTCSVT
GDSITSGYWNWIRKFPGKKLEYMGFISYSGDTYYNPSLKSRISITRDTS
KNQYHLQLNSVTSEDTATYYCARRNYVSNYGGWYFGVWGAGTTVTVSS

### Construction of 2G11-Vaccibody:

The 2G11 scFv construct (SEQ ID NO:33) was cloned into the plasmid pLNOH2 (Norderhaug, L. et al., 3 Immunol Methods, 1997) encoding a vaccibody framework at BsmI and BsiWI sites, giving the overall vaccibody format: 2G11 scFv-dimerisation domain-antigen. The dimerization domain was a human hinge region and CH3 as described in e.g. the International Patent Application with application number PCT/EP2012/076404 and the antigenic part was derived from Herpes Simplex virus 2, gD protein (SEQ ID NO:23). The construct also encoded a His-tag for easy detection in ELISA.

### Example 2

### Verification of 2G11-Vaccibody Targeting Chicken APCs

For the purpose of utilising 2G11 in a vaccibody vaccine directed towards chicken diseases, a prerequisite is that the novel vaccibody protein is able to bind chicken antigen presenting cells. 2G11 is recognising chicken MHCII. However, after isolation of only the Fv part of an intact antibody and transferred to a novel format, a verification of the sustainability of specificity is mandatory. The following example shows how the 2G11 vaccibody was produced as a protein and analysed for binding to chicken antigen presenting cells by flow cytometry.

### Transfection and Protein Production and Purification:

HEK293E cells were transiently transfected with the pLNOH2 2G11-vaccibody construct by using Lipofectamine™ 2000 (Invitrogen). Culture medium was harvested at day 3 and 6 and further concentrated by the use of Vivaspin 2 columns. The concentrated medium was tested by ELISA for protein production. Shortly, immunoplates were coated with anti-human CH3 antibody (MCA878, AbD Serotec). Dilutions of culture medium from transfected and un-transfected cells were added. 2G11-vaccibody proteins were further detected by adding anti-His tag antibody (ab27025, Abcam).

Isolation and Staining of Chicken PBMCs with 2G11-Vaccibody:

PBMCs from 24 ml chicken blood was isolated with Lymphoprep (Lympholyte®-M, Cedarlane) and the cells were adjusted to a final concentration of 10×10⁶ cells/ml. The cells were resuspended in 100 μl 10 μg/ml Fc Block and incubated for 15 min at 4° C. The cells were further stained for MHC II binding by adding 25 μl concentrated 2G11 vaccibodies followed by 10 μg/ml biotinylated anti-human IgG (05-4240, Invitrogen) and streptavidin-PE or corresponding isotype controls. A positive control was stained with 10 μg/ml 2G11 mAb (AH Diagnostics) and a negative medium control was included.

The cells were analyzed on a BD FACSCalibur by using the software CellQuest by gating for live lymphocytes, CD45 APC positive. The following histogram analysis is for MHC binding to CD45+ cells.

There is strong, clear binding of both the 2G11 mAb and 2G11 vaccibody to CD45+ leukocytes from chicken blood. Surprisingly the 2G11 vaccibody seems to bind the chicken cells better than the native 2G11 mAb. There is no binding of the negative controls; staining buffer, the non-targeting vaccibody or the isotype controls. The example verifies that 2G11 vaccibodies are able to target chicken antigen presenting cells.

### Example 3

### Immune Response Studies

Constructs according to the present invention are selected as vaccine candidates with their corresponding controls. As a negative control empty vector is utilized.

Different amounts of plasmid DNA of each candidate is administered by bodily injection, in the drinking water, as a spray for inhalation or injected in ovo. Chicken blood is drawn every week after vaccination for measurement of antigen specific antibodies. The antibody responses are calculated by ELISA.

### Example 4

### Preventive Effect

Chicken vaccinated with selected vaccibody vaccine candidates are challenged with pathological levels of the corresponding pathogen, being a virus or bacteria. Challenged chickens are monitored for disease development. The monitoring performed by measuring virus or bacterial levels as well as disease progression associated with the respective disease condition.

### Example 5

A DNA vaccine to be used may be prepared by GMP manufacturing of the plasmid vaccine according to regulatory authorities' guidelines, including GMP cell banking, GMP manufacturing of drug substance and drug product, ICH stability studies and Fill & Finish of the DNA vaccine. The DNA vaccine may be formulated by dissolving in a saline solution, such as 10 nM Tris, 1 mM EDTA or PBS pH7.4 at a concentration of 1-3 mg/ml or included in suitable viral vector systems. The vaccine may be administered either intra-dermal or intra-muscular, respiratorial, mucosal or via the GI tract, or in ovo.

### Example 6

The scFv fragment constituting the targeting unit of the described invention may be manipulated by means of altering affinity and specificity. The 2G11 scFv clone may be displayed as a fusion to phage particles and variations in the CDR-regions can be introduced by either erroneous PCR or specific PCR reactions with oligonucleotides introducing heterogeneity in the CDR regions. After generating a phage display library specific clones can be selected towards the specific target, i.e. a chicken MHC class II molecule. The selection process can be performed at different stringencies, such as low target concentration, high temperature or altered salt concentrations. Such conditions may develop a 2G11 scFv fragment with higher specificity and affinity towards the specific chicken MHCII molecule. Likewise, to obtain a 2G11 scFv fragment with a broader specificity, the phage display library can be selected towards a variety of MCHII molecules and selected clones harbouring specificity towards a variety of MHCII molecules can thus be selected. The manipulated scFV fragments can be utilised as new targeting units of the described invention.

1. A homodimeric protein of two identical amino acid chains, each amino acid chain comprising (1) optionally a signal peptide, (2) a targeting unit, (3) a dimerization motif, and (4) an antigenic unit, said targeting unit being a scFv fragment specifically binding avian MHC class II molecule, on avian cells.

2. The homodimeric protein according to claim 1, said targeting unit comprising an amino acid sequence having at least 80% sequence identity to the amino acid sequence of SEQ ID NO: 7, SEQ ID NO: 9, or SEQ ID NO: 11.

3. (canceled)

4. (canceled)

5. The homodimeric protein according to claim 1, wherein said antigenic unit is any protein unit such as a domain, short segment or a peptide or combinations of different protein units, derived from a pathogen or cancer related tissue.

6. The homodimeric protein according to claim 1, wherein said targeting unit, dimerization motif and antigenic unit in said amino acid chain are in the N-terminal to C-terminal order of targeting unit, dimerization motif and antigenic unit.

7. The homodimeric protein according to claim 1, wherein said targeting unit comprises an amino acid sequence having at least 85% sequence identity to the amino acid sequence of SEQ ID NO: 7, SEQ ID NO: 9, or SEQ ID NO: 11.

8. (canceled)

9. (canceled)

10. The homodimeric protein according to claim 1, wherein the dimerization motif comprises a hinge region or a shortened CH2 domain and optionally another domain that facilitate dimerization, optionally connected through a linker.

11-25. (canceled)

26. The homodimeric protein according to claim 1, wherein said antigenic unit comprises an antigenic unit suitable for preventive and/or therapeutic effect for poultry diseases.

27-32. (canceled)

33. An amino acid chain comprising (1) an optional signal peptide, (2) a targeting unit, (3) an optional dimerization motif, and (4) an antigenic unit, said targeting unit being a scFv fragment specifically binding chicken MHC class II molecule on avian cells.

34. (canceled)

35. A nucleic acid molecule, such as a DNA, encoding the amino acid chain according to claim 33.

36. (canceled)

37. (canceled)

38. A vector comprising the nucleic acid molecule according to claim 35.

39. (canceled)

40. The nucleic acid molecule according to claim 35 formulated for administration to the subject of, an avian population to induce production of the homodimeric protein in said subject.

41. The homodimeric protein according to claim 1 for use as a medicament.

42. A pharmaceutical composition comprising the homodimeric protein according to claim 1.

43. A host cell comprising the nucleic acid molecule according to claim 35.

44. A method for preparing a homodimeric protein or an amino acid chain, the method comprising
a) transfecting the nucleic acid molecule according to claim 35 into a cell population;
b) culturing the cell population;
c) collecting and purifying the homodimeric protein, or amino acid chain expressed from the cell population.

45. A method for preparing a vaccine, comprising an
immunologically effective amount of a nucleic acid molecule, the method comprising
a. preparing a nucleic acid molecule according to claim 35; and
b. dissolving the nucleic acid molecule obtained under step a) in a pharmaceutically acceptable carrier, diluent, or buffer.

46. A method for preparing a cell, producing a homodimeric protein or an amino acid chain, the method comprising
a. preparing a nucleic acid molecule according to claim 35;
b. activating in vitro the claim 1 with an immunologically effective amount of the nucleic acid molecule or vector prepared under step a); and
c. preparing the cells, such as antigen presenting cells obtained under step b) in a suitable diluent, such as a pharmaceutically acceptable carrier, diluent, or buffer.

47. A vaccine against a disease or condition in an animal, the vaccine comprising an immunologically effective amount of the homodimeric protein according to claim 1.

48. (canceled)

49. A method of treating or preventing a disease or condition in an animal, the method comprising administering to said animal in need thereof, the homodimeric protein according to claim 1.

50. (canceled)

51. The method according to claim 49, wherein the administration is performed intra-dermal or intra-muscular, respiratorial, mucosal, via the GI tract, or in ovo.
